SEQUENCE LISTING (8 sequences)

SEQ ID NO: 1 - protein, 15 aa, synthetic construct
AGAPEPAEPAQPGVY

SEQ ID NO: 2 - protein, 15 aa, synthetic construct
SETPEPKGPADPELY

SEQ ID NO: 3 - protein, 15 aa, synthetic construct
  VARIANT 1..3: note = X can be any amino acid
  VARIANT 7..8: note = X can be any amino acid
  VARIANT 11: note = X can be any amino acid
  VARIANT 13..14: note = X can be any amino acid
XXXPEPXXPAXPXXY

SEQ ID NO: 4 - protein, 9 aa, synthetic construct
  MOD_RES 1: note = tert-butyl
  MOD_RES 2: note = tert-butyl-oxy
  MOD_RES 3: note = tert-butyl
  MOD_RES 5: note = tert-butyl-oxy
  MOD_RES 7: note = tert-butoxycarbonyl
SETPEPKGP

SEQ ID NO: 5 - protein, 15 aa, synthetic construct
  MOD_RES 2: note = tert-butyl-oxy
  MOD_RES 4: note = tert-butyl-oxy
  MOD_RES 6: note = H-benzyl-phosphate
  MOD_RES 7: note = tert-butyl
  MOD_RES 8: note = tert-butyl-oxy
  MOD_RES 9: note = tert-butyl
  MOD_RES 11: note = tert-butyl-oxy
  MOD_RES 13: note = tert-butoxycarbonyl
ADPELYSETPEPKGP

SEQ ID NO: 6 - protein, 15 aa, synthetic construct
  MOD_RES 2: note = tert-butyl-oxy
  MOD_RES 4: note = tert-butyl-oxy
  MOD_RES 6: note = H-benzyl-phosphate
  MOD_RES 7: note = tert-butyl
  MOD_RES 8: note = tert-butyl-oxy
  MOD_RES 9: note = tert-butyl
  MOD_RES 11: note = tert-butyl-oxy
  MOD_RES 13: note = tert-butoxycarbonyl
ADPELYSETPEPKGP

SEQ ID NO: 7 - protein, 15 aa, synthetic construct
  MOD_RES 2: note = tert-butyl-oxy
  MOD_RES 4: note = tert-butyl-oxy
  MOD_RES 6: note = H-benzyl-phosphate
  MOD_RES 7: note = tert-butyl
  MOD_RES 8: note = tert-butyl-oxy
  MOD_RES 9: note = tert-butyl
  MOD_RES 11: note = tert-butyl-oxy
  MOD_RES 13: note = tert-butoxycarbonyl
ADPELYSETPEPKGP

SEQ ID NO: 8 - protein, 15 aa, synthetic construct
  MOD_RES 15: note = PO3H2
SETPEPKGPADPELY

(19) United States
(12) Patent Application Publication
(10) Pub. No.: US 2026/0070946 A1
(43) Pub. Date: Mar. 12, 2026

(54) TYR PEPTIDE COMPOSITIONS AND METHODS FOR USE

(71) Applicant: UNIVERSITY OF MIAMI, Miami, FL (US)

(72) Inventor: Karina Galoian, Miami, FL (US)

(21) Appl. No.: 19/107,327

(22) PCT Filed: Sep. 1, 2023
(86) PCT No.: PCT/US2023/073277

§ 371 Date: Feb. 27, 2025

Related U.S. Application Data
(60) Provisional application No. 63/374,251, filed on Sep. 1, 2022.
(60) Provisional application No. 63/522,636, filed on Jun. 22, 2023.

(51) Int. Cl. C07K 7/64; A61K 38/00; A61K 45/06; A61P 35/04; C07K 7/08

(52) U.S. Cl. CPC C07K 7/64; A61K 45/06; A61P 35/04; C07K 7/08; A61K 38/00

(57) ABSTRACT

The present disclosure, relates, in general to analogs of proline-rich polypeptide 1 (PRP-1) designated tyrosine peptides (TYR peptide) that are useful to treat cancer, such as sarcomas, carcinomas and leukemias or liquid cancers.

### CROSS REFERENCE TO RELATED APPLICATIONS

The present application claims the priority benefit of U.S. Provisional Patent Application No. 63/374,251, filed Sep. 1, 2022, and U.S. Provisional Patent Application No. 63/522,636, filed Jun. 22, 2023, herein incorporated by reference in their entireties.

### INCORPORATION BY REFERENCE OF SEQUENCE LISTING

The Sequence Listing, which is a part of the present disclosure, is submitted concurrently with the specification as an XML file. The name of the XML file containing the Sequence Listing is “58058_Seqlisting.XML”, which was created on Aug. 21, 2023 and is 20,594 bytes in size. The subject matter of the Sequence Listing is incorporated by reference herein in its entirety.

### FIELD OF THE DISCLOSURE

The present application is directed, in general, to analogs of proline rich polypeptide-1 (PRP-1) referred to as TYR peptides, compositions thereof and use to treat cancer, such as soft tissue, bone and liquid cancers.

### BACKGROUND

Proline rich polypeptide-1 (PRP-1), an antitumorigenic cytokine, is a fragment of the neurophysin-vasopressin-associated glycoprotein that is produced by hypothalamic neurosecretory cells. PRP-1 has been described as a potent immunomodulator which inhibits mTOR and cMyc, and suppresses cell cycle progression in high grade chondrosarcoma Proline-rich peptide (PRP)-1 has been shown to reduce sarcosphere formation and spheroid size in cell culture (Granger et al., Mol. Med. Rep. 22 (5): 3747-3758, 2020) hallmarks of metastatic cancer bestowed by cancer stem cells (CSCs) (Gibbs et al., Neoplasia, 7 (11): 967-76, 2005). CSCs have been demonstrated to contain a small subpopulation of self-renewing primitive/progenitor cells that maintain tumor cells thriving, and yet PRP-1 completely eradicated the stem cell population. PRP-1 Inhibits the cancer stem cell marker ALDH1A1 (Granger et al., supra). PRP-1 is also a mammalian target of rapamycin complex 1 (mTORC1) inhibitor and may act similarly as other mTOR inhibitors shown to have strong inhibitory effect in chondrosarcoma and breast adenocarcinoma (Galoian et al., Neurochemical Res. 36:812-818, 2011; Galoian et al., Tumor Biology, 32 (4): 745-51, 2011).

The innate immunity of Toll like receptors (TLR 1/2 and TLR 6) for PRP-1 in chondrosarcoma cells has been evaluated (Galoian et al., International Journal of Oncology (Spandidos publications), 52 (1): 139-154, 2018). PRP-1 was shown to inhibit most oncoproteins, onco-miRNas and upregulate tumor suppressor proteins, including desmosomal junction proteins, and miRNAs, depending on the pathways in which they are involved (Galoian et al. Molecular and Clinical Oncology 3:171-178, 2015; Galoian et al., Tumour Biol. 35 (3): 2335-41, 2014). PRP-1 is involved in fine epigenetic tuning of chondrosarcoma regulation. It has been demonstrated that targeting BAFF chromatin remodeling complexes by PRP-1 is contributing to decrease of chondrosarcoma cell-line growth and drastic decrease of cancer stem cells (Moran et al. Oncology Reports, 44:393-403, 2020).

### SUMMARY

Provided herein are peptide analogs of proline rich polypeptide-1 (PRP-1) which maintain the proline residue of the PRP-1 peptide and the C-terminal tyrosine residue, designated TYR peptides), and can be substituted at other residues with the PRP-1 peptide to provide novel peptides with anti-tumor function. The C-terminal tyrosine of the TYR peptides can be phosphorylated.

In one aspect, the disclosure provides a peptide analog of proline rich polypeptide-1 (PRP-1) (AGAPEPAEPAQPGVY, SEQ ID NO: 1) which is a tyrosine peptide (TYR peptide) having an amino acid sequence at least 70% identical to SETPEPKGPADPELY (SEQ ID NO: 2). In various embodiments, the tyrosine residue is a C-terminal amino acid of the peptide. In various embodiments, the TYR peptide is phosphorylated on a tyrosine residue.

In various embodiments, the TYR peptide comprises an amino acid sequence set out in X-X-X-PEP-X-X-PA-X-P-X-X-Y (SEQ ID NO: 3).

In various embodiments, the TYR peptide comprises the amino acid sequence SETPEPKGPADPELY (SEQ ID NO: 2). In various embodiments, the TYR peptide comprises the amino acid sequence SETPEPKGPADPELY-P0₃H₂.

In another embodiment, the TYR peptide is cyclized. In various embodiments, the cyclic TYR peptide is cyclized by linking the N-terminal amino acid with the C terminal amino acid. Provided is a cyclic TYR peptide having the sequence Cyclo(SETPEPKGPADPELY (PO₃H₂).

In various embodiments, the TYR peptide further comprises a pharmaceutically acceptable excipient, diluent or carrier.

Contemplated herein is a sterile composition comprising a TYR peptide as described herein.

Also contemplated is a nucleic acid encoding a TYR peptide as described herein.

In another aspect, the disclosure provides a method of treating a subject having cancer comprising administering a composition comprising a peptide analog of proline rich polypeptide-1 (PRP-1), wherein the peptide analog is a tyrosine peptide (TYR peptide) having an amino acid sequence at least 70% identical to SETPEPKGPADPELY (SEQ ID NO: 2).

In various embodiments, the cancer is carcinoma, sarcoma or leukemia. In various embodiments, the cancer is a sarcoma or leukemia. In various embodiments, the carcinoma is triple negative breast cancer.

In various embodiments, the disclosure provides a method of treating a sarcoma in a subject comprising administering a composition comprising a peptide analog of proline rich polypeptide-1 (PRP-1) which is a tyrosine peptide having an amino acid sequence at least 70% identical to SETPEPKGPADPELY (SEQ ID NO: 2).

In various embodiments, the sarcoma is selected from the group consisting of fibrosarcoma, sarcoma of the connective tissue, soft tissue sarcomas, leiomyosarcoma, malignant fibrous histiocytoma (MFH) and other undifferentiated pleomorphic sarcomas (PUS).

In various embodiments, the sarcoma is selected from the group consisting of fibrosarcoma, sarcoma of the connective tissue, soft tissue sarcomas, leiomyosarcoma, malignant fibrous histiocytoma (MFH) and other undifferentiated pleomorphic sarcomas (PUS) and osteosarcoma.

In various embodiments, the fibrosarcoma is infantile or congenital fibrosarcoma and adult-type fibrosarcoma.

In various embodiments, the fibrosarcoma is in the subject's thighs, lower leg, knees, arms, trunk, fat, muscles, tendons, nerves, joint tissue, or blood vessels.

In various embodiments, the disclosure provides a method of treating leukemia in a subject comprising administering a composition comprising a peptide analog of proline rich polypeptide-1 (PRP-1) which is a tyrosine peptide (TYR peptide) having an amino acid sequence at least 70% identical to SETPEPKGPADPELY (SEQ ID NO: 2).

In various embodiments, the leukemia is selected from the group consisting of acute lymphoblastic leukemia (ALL), acute myeloid leukemia (AML), mixed-lineage leukemia (MLL), chronic myeloid (or myelogenous) leukemia (CML), and chronic lymphocytic leukemia (CLL).

In various embodiments, the disclosure provides a method of treating carcinoma in a subject comprising administering a composition comprising a peptide analog of proline rich polypeptide-1 (PRP-1) which is a tyrosine peptide (TYR peptide) having an amino acid sequence at least 70% identical to SETPEPKGPADPELY (SEQ ID NO: 2).

In various embodiments, the carcinoma is triple negative breast cancer.

In various embodiments, the TYR peptide is administered in combination with one or more chemotherapeutic agents or second agents to treat cancer. In various embodiments, the chemotherapeutic agent is doxorubicin, prednisone, methotrexate, cyclophosphamide, doxorubicin hydrochloride (hydroxydaunorubicin), vincristine sulfate, bleomycin, vinblastine, dacarbazine bleomycin, etoposide, cisplatin, or a combination regimen such as CHOP [cyclophosphamide, doxorubicin hydrochloride (hydroxydaunorubicin), vincristine sulfate (ONCOVIN®), and prednisone], ABVD [doxorubicin (Adriamycin), bleomycin (Blenoxane®), vinblastine (Velban®) and dacarbazine (DTIC-Dome®)], and BEP [bleomycin (Blenoxane®), etoposide (Vepesid®) and cisplatin (Platinol®)].

If TYR peptides are administered in combination with a chemotherapeutic agent or second agent, the TYP peptide and or chemotherapeutic or second agent can be administered concurrently or sequentially. Concomitant or concurrent administration of two therapeutic agents does not require that the agents be administered at the same time or by the same route, as long as there is an overlap in the time period during which the agents are exerting their therapeutic effect. Simultaneous or sequential administration is contemplated, as is administration on different days or weeks. It is further contemplated that the therapeutics are administered in a separate formulation and administered concurrently or concomitantly, with concurrently referring to agents given within 30 minutes of each other. Prior administration refers to administration of a therapeutic within the range of one week prior to treatment with a carrier particle, up to 30 minutes before administration of a carrier particle. Subsequent administration is meant to describe administration from 30 minutes after treatment up to one week after administration.

In various embodiments, the one or more chemotherapeutic or second agent is administered prior to, concurrently with or subsequent to administration of the TYR peptide described herein. In various embodiments, the chemotherapeutic or second agent is administered 0.5, 1, 2, 4, 5, 8, 10, 12, 16 or 24 hours prior to administration of the TYR peptide. In various embodiments, the chemotherapeutic or second agent is administered 1, 2, 3, 4, 5, 6, or 7 days prior to administration of the TYR peptide. In various embodiments, the chemotherapeutic or second agent is administered 1, 2, 3, or 4 weeks prior to administration of TYR peptide. In various embodiments, the chemotherapeutic or second agent is administered concurrently with the TYR peptide. In various embodiments, the chemotherapeutic or second agent is administered 0.5 1, 2, 4, 5, 8, 10, 12, 16 or 24 hours subsequent to administration of the TYR peptide. In various embodiments, the chemotherapeutic or second agent is administered 1, 2, 3, 4, 5, 6, or 7 days subsequent to administration of the TYR peptide. In various embodiments, the chemotherapeutic or second agent is administered 1, 2, 3, or 4 weeks subsequent to administration of the TYR peptide.

In various embodiments, the TYR peptide decreases growth and/or proliferation of sarcoma cells, leukemia cells and/or cancer stem cells (CSC). In various embodiments, the TYR peptide is cytotoxic for cancer stem cells.

In various embodiments, the TYR peptide reduces tumor size or tumor volume in the subject. In various embodiments, the methods reduce tumor size or tumor volume by 10%, 15%, 20%, 25%, 30%, 35%, 40%, 45%, 50%, 55%, 60%, 65%, 70%, 75%, 80%, 85%, 90%, 95% or 100%.

In various embodiments, the TYR peptide reduces incidence of metastasis or reduces incidence of tumor recurrence in the subject. In various embodiments, the incidence of metastasis or tumor recurrence is reduced by 10%, 15%, 20%, 25%, 30%, 35%, 40%, 45%, 50%, 55%, 60%, 65%, 70%, 75%, 80%, 85%, 90%, 95% or 100%.

In various embodiments, the TYR peptide is administered to a subject having cancer who is resistant to previous treatments or has recurrence of cancer after an initial therapeutic treatment.

In various embodiments, the TYR peptide is administered with a second agent or standard of care therapy. In various embodiments, the second agent or standard of care is a chemotherapeutic, a cytotoxic agent, a radionuclide, an immunotherapeutic, radiation therapy, or combinations thereof.

In various embodiments, the TYR peptide useful in the methods comprises an amino acid sequence set out in X-X-X-PEP-X-X-PA-X-P-X-X-Y (SEQ ID NO: 3). In various embodiments, the TYR peptide is phosphorylated on the tyrosine residue. In various embodiments, the TYR peptide comprises the amino acid sequence SETPEPKGPADPELY (SEQ ID NO: 2). In various embodiments, the TYR peptide comprises the amino acid sequence SETPEPKGPADPELY-P0₃H₂. In various embodiments, the TYR peptide is cyclized. In various embodiments, the TYR peptide is a cyclic TYR peptide having the sequence Cyclo(SETPEPKGPADPELY (PO₃H₂) (SEQ ID NO: 2).

In various embodiments, the peptide analog is administered on a period basis, for example, hourly, daily, twice weekly, weekly, every 2 weeks, every 3 weeks, monthly, once every two months or at a longer interval.

Also contemplated is a composition comprising any of the foregoing TYR peptides or compositions of the disclosure, or use thereof in preparation of a medicament, for treatment of cancer as described herein. Syringes, e.g., single use or pre-filled syringes, sterile sealed containers, e.g., vials, bottle, vessel, and/or kits or packages comprising any of the foregoing peptides or compositions, optionally with suitable instructions for use, are also contemplated.

It is understood that each feature or embodiment, or combination, described herein is a non-limiting, illustrative example of any of the aspects of the invention and, as such, is meant to be combinable with any other feature or embodiment, or combination, described herein. For example, where features are described with language such as “one embodiment”, “some embodiments”, “certain embodiments”, “further embodiment”, “specific exemplary embodiments”, and/or “another embodiment”, each of these types of embodiments is a non-limiting example of a feature that is intended to be combined with any other feature, or combination of features, described herein without having to list every possible combination. Such features or combinations of features apply to any of the aspects of the invention. Where examples of values falling within ranges are disclosed, any of these examples are contemplated as possible endpoints of a range, any and all numeric values between such endpoints are contemplated, and any and all combinations of upper and lower endpoints are envisioned.

### BRIEF DESCRIPTION OF THE DRAWINGS

FIG. 1 shows the inhibitory effect of TYR peptide on proliferation of high grade primary pleomorphic undifferentiated sarcoma (malignant fibrous histiocytoma) of leiomyosarcoma phenotype. Inhibition with Tyr peptide (1 μg/ml) reached 90% and with Tyr peptide (10 μg/ml)-74% in comparison with untreated control.

FIGS. 2A-2B show the TYR peptide inhibitory effect on human acute lymphoblastic leukemia (ALL) cell line (1γ-μg/ml) at 48 hr (FIG. 2A) and 72 hrs (FIG. 2B).

FIGS. 3A-3B show dose response effect with TYR peptide analog (TPA) 1 in human fibrosarcoma, with the maximum effect with 40 μg/ml (FIG. 3A). FIG. 3B visually demonstrates destruction of cancer stem cell spheroids in dose response manner, with the maximum effect with 40 μ/ml.

FIG. 4 depicts a cytotoxicity assay demonstrating dose response decrease in viability of fibrosarcoma spheroids with increased concentration of TPA peptide.

FIG. 5 shows results of a self-renewal assay with 3D fibrosarcoma and TPA treatment depicting the destruction and shrinkage of most spheroids with TPA 20 μg/ml in comparison to untreated control, and prevention of spheroid formation with 40 μg/ml.

FIG. 6 illustrates IVIS fluorescence imaging data showed decreased growth in the TPA Group mice compared to the Control through Day 14.

FIG. 7 shows tumor volume decreased somewhat in the TPA Group mice compared to the Control through Day 18.

FIG. 8 shows quantitative IVIS imaging of tumor growth after TPA+Doxo treatment compared to Doxo alone in relapsed mice. Days represent days from the first Doxo treatment (on day 1).

FIG. 9 shows median survival for mice receiving combination treatment, doxorubicin treatment and control (PBS) was 35, 28, 21 days, respectively. 10 mice per group.

FIG. 10 shows cytotoxicity in cardiomyocytes (AC16) after corresponding treatments. Statistical analysis One-way ANOVA following by Dunnett's test, ***P≤0.0001.

FIG. 11 shows the concentration-response inhibition of the osteosarcoma cell lines OSA (FIG. 11A), KHOS (FIG. 11B), and MNNG (FIG. 11C). The inhibition/viability was >50% after 48h of incubation with TPA. T-test *P≤0.05 and **P≤0.01

FIGS. 12A-12B show there is a dose-response effect of TPA on the proliferation of triple-negative human breast cancer cells (MDA-MB-231) in WST-1 assay. (FIG. 12A) % Response and IC50. (FIG. 12B) Cell growth %. Statistical analysis: ANOVA followed by Dunnett's multiple comparison test. *P≤0.001, n=3

FIGS. 13A-13D show gene expression after TPA treatment of cells in culture. (FIG. 13A). Gene expression of mRNA sequences coding for Nanog in 3D lysates treated with TPA (20 μg/mL) and control. Statistical Analysis T-Test **p<0.0001. (FIG. 13B) Gene expression of mRNA sequences coding for EZH2 in 3D lysates treated with TPA (20 μg/mL), and control. Statistical analysis, T-Test **p<0.0001. (FIG. 13C) Gene expression of mRNA sequences coding for ALDHA in 3D lysates treated with TPA (20 μg/mL), and control. Statistical Analysis T-Test *p≤0.05. (FIG. 13D) Dose response inhibition of Nanog protein expression in 3D fibrosarcoma cells (HT1080) by TPA (40 μg/ml), Statistical analyses ANOVA test *P<0.05. n=2. All results were normalized by the expression of GAPDH mRNA and are presented as the fold-difference compared with that of the control condition.

FIG. 14A shows the structure of cyclic TYR peptide. FIG. 14B shows the potency of cyclic TYR peptide compared to non-cyclic TYR peptide in a rapid cell proliferation assay. ANOVA following by Dunnett's test, *p≤0.05, **p≤0.001.

### DETAILED DESCRIPTION

The present application relates to the discovery that PRP-1 peptide analogs, termed TYR peptides, are useful in the treatment of cancers, such as soft tissue sarcoma and leukemia. TYR peptides described herein possess unique properties compared to PRP-1 peptides in their ability to kill cancer stem cells and inhibit growth of certain cancer types.

### Definitions

The term “PRP-1 analog” or “TYR peptide” as used herein refers to a peptide sequence comprising a string of 15-20 amino acids that preserve multiple proline residues as in the native PRP-1 sequence AGAPEPAEPAQPGVY (SEQ ID NO: 1), but having altered amino acid sequence, e.g., X-X-X-PEP-X-X-PA-X-P-X-X-Y (SEQ ID NO: 3), and having a tyrosine on the C-terminus. A “phosphorylated TYR peptide” is a TYR peptide in which the tyrosine residue on the C-terminus of the peptide is phosphorylated.

The term “cytotoxic” refers to an agent's ability to kill or eliminate cells, e.g., cancer cells, and cancer stem cells, after the cell comes into contact with the agent. Cytotoxic is different than cytostatic, which refers to slowing the growth of a cell without necessarily killing the cell.

The term “derivative” when used in connection with peptide analogs of the present disclosure refers to polypeptides chemically modified by such techniques as ubiquitination, conjugation to therapeutic or diagnostic agents, labeling (e.g., with radionuclides or various enzymes), covalent polymer attachment such as PEGylation (derivatization with polyethylene glycol) and insertion or substitution by chemical synthesis of amino acids such as ornithine, which do not normally occur in human proteins. Derivatives retain the binding properties of underivatized molecules of the disclosure.

“Detectable moiety” or “label” refers to a composition detectable by spectroscopic, photochemical, biochemical, immunochemical, or chemical means. For example, useful labels include 32P, 35S, fluorescent dyes, electron-dense reagents, enzymes (e.g., as commonly used in an ELISA), biotin-streptavadin, dioxigenin, haptens and proteins for which antisera or monoclonal antibodies are available, or nucleic acid molecules with a sequence complementary to a target. The detectable moiety often generates a measurable signal, such as a radioactive, chromogenic, or fluorescent signal, that can be used to quantitate the amount of bound detectable moiety in a sample.

The term “subject” encompasses mammals and non-mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. The term does not denote a particular age or gender.

The term “therapeutically effective amount” is used herein to indicate the amount of target-specific composition of the disclosure that is effective to ameliorate or lessen symptoms or signs of disease to be treated.

The terms “treat”, “treated”, “treating” and “treatment”, as used with respect to methods herein refer to eliminating, reducing, suppressing or ameliorating, either temporarily or permanently, either partially or completely, a clinical symptom, manifestation or progression of an event, disease or condition. Such treating need not be absolute to be useful.

### TYR Peptides

TYR peptides are analogs of PRP-1 peptide that comprise multiple proline residues and a tyrosine residue on the C-terminus, compared to the PRP-1 peptide (AGAPEPAEPAQPGVY, SEQ ID NO: 1). TYR peptides contemplated herein have an amino acid sequence at least 70% identical to SETPEPKGPADPELY (SEQ ID NO: 2). In various embodiments, the TYR peptide is cyclized. In various embodiments, the TYR peptide is a cyclic TYR peptide having the sequence Cyclo(SETPEPKGPADPELY (PO₃H₂).

In various embodiments, the TYR peptide comprises an amino acid sequence set out in X-X-X-PEP-X-X-PA-X-P-X-X-Y (SEQ ID NO: 3). TYR peptides include optionally up to 1, 2, 3, 4, 5, 6, 7, or 8 modifications (deletions, additions and/or substitutions) compared to SEQ ID NO: 2, and can be at positions designated X in SEQ ID NO: 3. The amino acid substitutions may be conservative substitutions or other substitutions described herein or known in the art.

“Conservative substitution” refers to substitution of an amino acid in a polypeptide with a functionally, structurally or chemically similar natural or unnatural amino acid. In one embodiment, the following groups each contain natural amino acids that are conservative substitutions for one another:
(1), Alanine (A) Serine(S), Threonine (T);
(2) Aspartic acid (D), Glutamic acid (E);
(3) Asparagine (N), Glutamine (Q);
(4) Arginine (R), Lysine (K);
(5) Isoleucine (I), Leucine (L), Methionine (M), Valine (V); and
(6) Phenylalanine (F), Tyrosine (Y), Tryptophan (W).

In another embodiment, the following groups each contain natural amino acids that are conservative substitutions for one another:
(1) Glycine (G), Alanine (A);
(2) Aspartic acid (D), Glutamic acid (E);
(3) Asparagine (N), Glutamine (Q);
(4) Arginine (R), Lysine (K);
(5) Isoleucine (I), Leucine (L), Methionine (M), Valine (V), Alanine (A);
(6) Phenylalanine (F), Tyrosine (Y), Tryptophan (W); and
(7) Serine(S), Threonine (T), Cysteine (C).

In a further embodiment, amino acids may be grouped as set out below.
(1) hydrophobic: Met, Ala, Val, Leu, Ile, Phe, Trp;
(2) neutral hydrophilic: Cys, Ser, Thr, Asn, Gln;
(3) acidic: Asp, Glu;
(4) basic: His, Lys, Arg;
(5) residues that influence backbone orientation: Gly, Pro; and
(6) aromatic: Trp, Tyr, Phe, His.

In one embodiment, the peptides described herein are generated via recombinant means, using a polynucleotide encoding a TYR peptide. The disclosure thus encompasses polynucleotides encoding any of the TYR peptides described herein, host cells or vectors comprising such polynucleotides, optionally linked to expression control sequences, and methods of using such polynucleotides, vectors or host cells to produce TYR peptides of the disclosure. TYR peptides expressed by such polynucleotides may be produced by methods including growing host cells in culture medium under conditions suitable for expression of the polynucleotide encoding a CNP variant, and isolating the expression product from the host cells or culture medium. Actual expression products may vary slightly from the encoded protein product depending on any post-translational processing.

“Polynucleotide” refers to a polymer composed of nucleotide units. Polynucleotides include naturally occurring nucleic acids, such as deoxyribonucleic acid (“DNA”) and ribonucleic acid (“RNA”) as well as nucleic acid analogs. The term “nucleic acid” typically refers to large polynucleotides. The term “oligonucleotide” typically refers to short polynucleotides, generally no greater than about 50 nucleotides. It will be understood that when a nucleotide sequence is represented by a DNA sequence (i.e., A, T, G, C), the nucleotide sequence also encompasses an RNA sequence (i.e., A, U, G, C) in which “U” replaces “T.” “cDNA” refers to a DNA that is complementary or identical to an mRNA, in either single stranded or double stranded form.

It is contemplated that the TYR peptide is phosphorylated on the tyrosine residue. In various embodiments, the TYR peptide is phosphorylated on the tyrosine residue at the C-terminus of the peptide.

Contemplated herein is a TYR peptide describe herein, further comprising a pharmaceutically acceptable excipient, diluent or carrier. As such, the disclosure provides a sterile composition comprising a TYR peptide described herein.

### Derivatives

As stated above, derivative, when used in connection with peptides of the disclosure, refers to peptides chemically modified by such techniques as ubiquitination, labeling (e.g., with radionuclides or various enzymes), covalent polymer attachment such as PEGylation (derivatization with polyethylene glycol) and insertion or substitution by chemical synthesis of amino acids such as ornithine. Derivatives of the peptides disclosed herein are also useful as therapeutic agents and may be used in the methods herein.

The conjugated moiety can be incorporated in or attached to a peptide either covalently, or through ionic, van der Waals or hydrogen bonds, e.g., incorporation of radioactive nucleotides, or biotinylated nucleotides that are recognized by streptavidin.

Polyethylene glycol (PEG) may be attached to the peptide to provide a longer half-life in vivo. The PEG group may be of any convenient molecular weight and may be linear or branched. The average molecular weight of the PEG will preferably range from about 2 kiloDalton (“kD”) to about 100 kDa, more preferably from about 5 kDa to about 50 kDa, most preferably from about 5 kDa to about 10 kDa. The PEG groups will generally be attached to the peptides of the disclosure via acylation or reductive alkylation through a natural or engineered reactive group on the PEG moiety (e.g., an aldehyde, amino, thiol, or ester group) to a reactive group on the peptide (e.g., an aldehyde, amino, or ester group). Addition of PEG moieties to peptides can be carried out using techniques well-known in the art. See, e.g., International Publication No. WO 96/11953 and U.S. Pat. No. 4,179,337.

Ligation of the peptide with PEG usually takes place in aqueous phase and can be easily monitored by reverse phase analytical HPLC. The PEGylated substances are purified by preparative HPLC and characterized by analytical HPLC, amino acid analysis and laser desorption mass spectrometry.

### Methods of Use

The TYR peptides and analogs described herein are useful to treat cancers, such as sarcomas, carcinomas and leukemias or other liquid cancers.

Rare cancers are defined as malignancies with an incidence of <6 per 100,000 per year. For example, 16,000 patients are diagnosed in the United States each year. Sarcoma is cancer of the connective tissue, found in the bones, fat tissue, cartilage or muscles. Among Sarcomas there are types which are considered as Ultrarare. Ultrarare sarcomas were defined as those with an incidence of approximately ≤1 per 1,000,000, z according to the consensus paper written by sarcoma world experts (Stacchiotti, et al Ultra-Rare Sarcomas: A Consensus Paper From the Connective Tissue Oncology Society Community of Experts on the Incidence Threshold and the List of Entities Cancer. 2021 Aug. 15; 127 (16): 2934-2942). According to these classifications the chondrosarcoma and fibrosarcoma presented in this application are ultrarare diseases.

Malignant fibrous histiocytoma (MFH) has been regarded as the most common type of soft tissue sarcoma of late adulthood. The latest World Health Organization classification of soft tissue tumors regards MFH as a synonym for undifferentiated pleomorphic sarcomas (PUS) and, therefore, the diagnosis of MFH has become a diagnosis of exclusion and is reserved for tumors showing no other line of differentiation. It is now widely accepted that the morphological pattern of pleomorphic MFH may be shared by a wide variety of poorly differentiated malignant mesenchymal This study was carried out to determine and compare the frequency of allelic imbalance at different loci on chromosome 9p in MFH. Both genes are located at 9p21 and encode nuclear proteins that have been shown to block cell cycle progression at the G1-S transition. Therefore, the diagnosis of MFH is one of exclusion following thorough sampling and the use of ancillary techniques. The genetic profile of MFH is difficult to evaluate because of the lack of specific diagnostic features or immunohistochemistry used throughout the years. However, it has been reported that MFH tumors have similar comparative genomic hybridization imbalances and similar genome expression patterns (Lee et al., Br J Cancer 88:510-515, 2003). MFH share similar allelic imbalance at 9p, particularly at D9S230. While alterations at this locus may represent changes common to pleomorphic sarcomas, they were rare in synovial sarcomas and malignant peripheral nerve sheath tumors. This would suggest that this locus may harbor a tumor suppressor gene that is involved in the genesis of MFH It further supports the hypothesis that MFH are related and that MFHs may represent a morphological pathway in tumor progression of LMSs. Although MFH were once thought to be a separate member of the soft tissue sarcoma family, observations support the hypothesis that MFH are a morphologic modulation in the tumoral progression of other sarcomas. Results suggest that MFH do not represent a specific sarcomatous entity, but may instead represent a common oncogenic pathway and possibly other soft tissue sarcomas (STS) (Sabah et al. Virchows Arch. 446 (3): 251-8, 2005; Man et al., Curr Hematol Malig Rep. 12 (3): 197-206, 2017).

Cancer stem cells (CSC) are a subpopulation of tumor cells that can drive tumor initiation and can cause relapses (Lia Walcher, Front Immunol, 11:1280. 2020). Implicit in this power is the ability of such cells to divide asymmetrically, yielding daughters that remain as CSCs (the trait of self-renewal) as well as daughters that differentiate into the neoplastic cells forming the bulk of the tumor (Diwakar et al., Nat Rev Drug Discov. 13 (7): 497-512, 2014). The number of molecules screened and entered for clinical trial for CSC-targeting is comparatively low, compared to other drugs. Biological activities of CSCs are regulated by several pluripotent transcription factors, such as NANOG, MYC, (Yang et al., Signal Transduct Target Ther. 5:8, 2020). These factors are inhibited by PRP-1 as was reported previously (Moran A et al, 2020, supra). It is hypothesized herein that anti-CSC therapy should eliminate the pool of cancer cells that are intrinsically resistant to conventional therapies, while a concomitantly administered conventional agent would eliminate the non-CSC cells, which are known to be susceptible to existing cytotoxic therapies.

Greater tumor shrinkage favors independent clonal expansion (Leonid Hanin, Appl. Math. Stat., 6:27, 2020). The recent findings explain numerous clinical cases where initial tumor shrinkage to undetectable levels was promptly followed by an aggressive tumor recurrence. They also suggest that indiscriminate use of tumor shrinkage as surrogate endpoint in clinical trials should be discouraged. This factor may bring about treatment failure and shorten disease-free, progression-free, and overall survival. Due to the treatment-induced decrease in cell density surviving cancer cells enjoy a greater supply of space, nutrients, oxygen, and growth factors, which is likely to decrease the rates of their death and quiescence and thus to increase their net proliferation rates. Furthermore, this effect is likely to be more pronounced for larger tumor shrinkage. In radiation therapy, the phenomenon of treatment-induced accelerated tumor repopulation has been recognized long ago.

TPA inhibits in a statistically significant manner developmental homeotic HOXC13 proteins, which are part of human replication origins. Hox proteins are involved in cellular specification and identity. These proteins are known to have a homeodomain that binds DNA at specifically recognized binding sites and transcriptionally activates (or represses) their target genes, (Comelli et al., Cell Cycle. 2009 8 (3): 454-9). Hox proteins have also been found to have a role in tumor development (Botti et al., Cancers 2019 11 (5): 699).

It is hypothesized that extensive shrinkage of the primary tumor could accelerate metastasis; moreover, one would expect this effect to be proportional to the extent of tumor shrinkage. In the case of primary tumor resection, rapid outgrowth of metastases in animal models was directly observed, and since then, in countless experimental studies and clinical cases (Chiarella et al., Cancer Letters. (2012) 324:133-41).

Provided herein is a method of treating cancer in a subject comprising administering a composition comprising a peptide analog of proline rich polypeptide-1 (PRP-1) which is a tyrosine peptide (TYR peptide) having an amino acid sequence at least 70% identical to SETPEPKGPADPELY (SEQ ID NO: 2).

Also provided is a method of treating a sarcoma in a subject comprising administering a composition comprising a peptide analog of proline rich polypeptide-1 (PRP-1) which is a tyrosine peptide (TYR peptide) having an amino acid sequence at least 70% identical to SETPEPKGPADPELY (SEQ ID NO: 2).

In various embodiments, the sarcoma is selected from the group consisting of fibrosarcoma, sarcoma of the connective tissue, soft tissue sarcomas, leiomyosarcoma, malignant fibrous histiocytoma (MFH) and other undifferentiated pleomorphic sarcomas (PUS). In various embodiments, the sarcoma is selected from the group consisting of fibrosarcoma, sarcoma of the connective tissue, soft tissue sarcomas, leiomyosarcoma, malignant fibrous histiocytoma (MFH) and other undifferentiated pleomorphic sarcomas (PUS) and osteosarcoma.

In various embodiments, the fibrosarcoma is infantile or congenital fibrosarcoma and adult-type fibrosarcoma. In various embodiments, the fibrosarcoma is in the subject's thighs, lower leg, knees, arms, trunk, fat, muscles, tendons, nerves, joint tissue, or blood vessels.

Also provided is a method of treating a leukemia in a subject comprising administering a composition comprising a peptide analog of proline rich polypeptide-1 (PRP-1) which is a tyrosine (TYR) peptide having an amino acid sequence at least 70% identical to SETPEPKGPADPELY (SEQ ID NO: 2).

Exemplary leukemias include acute lymphocytic leukemia (ALL), acute myeloid leukemia (AML), mixed-lineage leukemia (MLL), chronic myeloid (or myelogenous) leukemia (CML), and chronic lymphocytic leukemia (CLL).

In various embodiments, the disclosure provides a method of treating carcinoma in a subject comprising administering a composition comprising a peptide analog of proline rich polypeptide-1 (PRP-1) which is a tyrosine peptide (TYR peptide) having an amino acid sequence at least 70% identical to SETPEPKGPADPELY (SEQ ID NO: 2). In various embodiments, the carcinoma is triple negative breast cancer.

It is contemplated that administration of the TYR peptide to a subject decreases growth and/or proliferation of sarcoma cells, leukemia cells and/or cancer stem cells (CSC). In another embodiment, the TYR peptide does not cause significant negative effects or toxicity on normal cells.

It is contemplated that administration of the TYR peptide to a subject decreases growth and/or proliferation of sarcoma cells, leukemia cells and/or cancer stem cells (CSC) or breast cancer cells.

For example, the TYR peptide is cytotoxic for cancer stem cells (CSC). In various embodiments, the TYR peptide eliminates 15% or more of cancer stem cells (CSC) in a subject. In various embodiments, the TYR peptide eliminates 15%, 20%, 25%, 30%, 35%, 40%, 45%, 50%, 55%, 60%, 65%, 70%, 75%, 80%, 85%, 90%, 95% or 100% of cancer stem cells (CSC) in a subject.

In various embodiments, the TYR peptide reduces tumor size or tumor volume in the subject. In various embodiments, the methods reduce tumor size or tumor volume by 10%, 15%, 20%, 25%, 30%, 35%, 40%, 45%, 50%, 55%, 60%, 65%, 70%, 75%, 80%, 85%, 90%, 95% or 100%.

In various embodiments, the TYR peptide reduces incidence of metastasis or reduces incidence of tumor recurrence in the subject. In various embodiments, the TYR peptide is administered to a subject having cancer who is resistant to previous treatments or has recurrence of cancer after an initial therapeutic treatment.

In various embodiments, the TYR peptide is administered with a second agent or standard of care therapy. In various embodiments, the second agent or standard of care is a chemotherapeutic, a cytotoxic agent, a radionuclide, an immunotherapeutic, radiation therapy, or combinations thereof.

In various embodiments, the subject is non-responsive to previous cancer therapies or developed drug resistance to other cancer therapies.

In various embodiments, the TYR peptide is phosphorylated on the tyrosine residue. In various embodiments, the tyrosine residue is a C-terminal amino acid of the peptide. In various embodiments, the TYR peptide comprises the amino acid sequence SETPEPKGPADPELY (SEQ ID NO: 2). In various embodiments, the TYR peptide comprises the amino acid sequence SETPEPKGPADPELY-P0₃H₂. In various embodiments, the TYR peptide is cyclized. In various embodiments, the TYR peptide is a cyclic TYR peptide having the sequence Cyclo(SETPEPKGPADPELY (PO₃H₂).

### Assays

Tumor growth can be monitored for several days by bioluminescence before the tumor size becomes palpable or measurable by traditional physical means. This rapid monitoring can provide insight into early events in tumor development or lead to shorter experimental procedures. Tumor cell death and necrosis due to hypoxia or drug treatment is indicated early by a reduction in the bioluminescent signal. This cell death might not be accompanied by a reduction in tumor size as measured by physical means. The ability to see early events in tumor necrosis has significant impact on the selection and development of therapeutic agents (Lim et al., J Vis Exp, 2009; (26): 1210).

Proliferation experiments are useful to determine the extent of growth reduction and cytotoxicity of the TYR peptide on different cancer cell types.

Effects of TYR peptide therapy on cancer cells or cancer stem cells can also be measured by levels of cancer stem cells factors such as NANOG or MYC (Yang L, et al. Signal Transduct Target Ther 5:8, 2020) or by analysis of ALDH or ALDH isozyme expression (Amrutha Mohan, Front Oncol 11:669250, 2021).

### Combination Therapy

A TYR peptide of the present disclosure is administered with a second agent or standard of care treatment, and the combination is useful to treat a cancer as described herein. For example, the patient may also be administered surgical therapy, chemotherapy, a cytotoxic agent, photodynamic therapy, radiation therapy, or combinations thereof where appropriate.

It is contemplated that one or more peptides of the present disclosure and the second agent may be given simultaneously, in the same formulation. It is further contemplated that the peptides are administered in a separate formulation and administered concurrently, with concurrently referring to agents given within 30 minutes of each other.

In another aspect, a peptide analog is administered prior to administration of the second agent. Prior administration refers to administration of peptide within the range of one week prior to treatment with the second agent, up to 30 minutes before administration of the second agent. It is further contemplated that a peptide is administered subsequent to administration of second agent. Subsequent administration is meant to describe administration from 30 minutes after peptide treatment up to one week after peptide administration.

A cytotoxic agent refers to a substance that inhibits or prevents the function of cells and/or causes destruction of cells. The term is intended to include radioactive isotopes (e.g., I¹³¹, I¹²⁵, Y⁹⁰ and Re¹⁸⁶), chemotherapeutic agents, and toxins such as enzymatically active toxins of bacterial, fungal, plant or animal origin or synthetic toxins, or fragments thereof. A non-cytotoxic agent refers to a substance that does not inhibit or prevent the function of cells and/or does not cause destruction of cells. A non-cytotoxic agent may include an agent that can be activated to be cytotoxic. A non-cytotoxic agent may include a bead, liposome, matrix or particle (see, e.g., U.S. Patent Publications 2003/0028071 and 2003/0032995 which are incorporated by reference herein). Such agents may be conjugated, coupled, linked or associated with a peptide according to the disclosure.

Chemotherapeutic agents contemplated for use with the peptides of the present disclosure include, but are not limited to those listed in Table I:

TABLE I

| | |
|---|---|
| Alkylating agents | Natural products |
| Nitrogen mustards | Antimitotic drugs |
| Mechlorethamine | Taxanes |
| cyclophosphamide | paclitaxel |
| ifosfamide | Vinca alkaloids |
| melphalan | vinblastine (VLB) |
| chlorambucil | vincristine |
| Nitrosoureas | vinorelbine |
| carmustine (BCNU) | Taxotere ® (docetaxel) |
| lomustine (CCNU) | Estramustine |
| semustine (methyl-CCNU) | estramustine phosphate |
| Ethylenimine/Methyl-melamine | Epipodophylotoxins |
| thriethylenemelamine (TEM) | etoposide |
| triethylene thiophosphoramide | teniposide |
| (thiotepa) | Antibiotics |
| hexamethylmelamine | actimomycin D |
| (HMM, altretamine) | daunomycin (rubido-mycin) |
| Alkyl sulfonates | doxorubicin (adria-mycin) |
| busulfan | mitoxantroneidarubicin |
| Triazines | bleomycin |
| dacarbazine (DTIC) | splicamycin (mithramycin) |
| Antimetabolites | mitomycinC |
| Folic Acid analogs | dactinomycin |
| methotrexate | aphidicolin |
| Trimetrexate | Enzymes |
| Pemetrexed | L-asparaginase |
| (Multi-targeted antifolate) | L-arginase |
| Pyrimidine analogs | Radiosensitizers |
| 5-fluorouracil | metronidazole |
| fluorodeoxyuridine | misonidazole |
| gemcitabine | desmethylmisonidazole |
| cytosine arabinoside | pimonidazole |
| (AraC, cytarabine) | etanidazole |
| 5-azacytidine | nimorazole |
| 2,2′-difluorodeoxy-cytidine | RSU 1069 |
| Purine analogs | E09 |
| 6-mercaptopurine | RB 6145 |
| 6-thioguanine | SR4233 |
| azathioprine | nicotinamide |
| 2′-deoxycoformycin | 5-bromodeozyuridine |
| (pentostatin) | 5-iododeoxyuridine |
| erythrohydroxynonyl- | bromodeoxycytidine |
| adenine (EHNA) | Miscellaneous agents |
| fludarabine phosphate | Platinium coordination complexes |
| 2-chlorodeoxyadenosine | cisplatin |
| (cladribine, 2-CdA) | Carboplatin |
| Type I Topoisomerase Inhibitors | oxaliplatin |
| camptothecin | Anthracenedione |
| topotecan | mitoxantrone |
| irinotecan | Substituted urea |
| Biological response modifiers | hydroxyurea |
| G-CSF | Methylhydrazine derivatives |
| GM-CSF | N-methylhydrazine (MIH) |
| Differentiation Agents | procarbazine |
| retinoic acid derivatives | Adrenocortical suppressant |
| Hormones and antagonists | mitotane (o,p′- DDD) |
| Adrenocorticosteroids/ antagonists | ainoglutethimide |
| prednisone and equivalents | Cytokines |
| dexamethasone | interferon (α, β, γ) |
| ainoglutethimide | interleukin-2 |
| Progestins | Photosensitizers |
| hydroxyprogesterone caproate | hematoporphyrin derivatives |
| medroxyprogesterone acetate | Photofrin ® |
| megestrol acetate | benzoporphyrin derivatives |
| Estrogens | Npe6 |
| diethylstilbestrol | tin etioporphyrin (SnET2) |
| ethynyl estradiol/equivalents | pheoboride-a |
| Antiestrogen | bacteriochlorophyll-a |
| tamoxifen | naphthalocyanines |
| Androgens | phthalocyanines |
| testosterone propionate | zinc phthalocyanines |
| fluoxymesterone/equivalents | Radiation |
| Antiandrogens | X-ray |
| flutamide | ultraviolet light |
| gonadotropin-releasing | gamma radiation |
| hormone analogs | visible light |
| leuprolide | infrared radiation |
| Nonsteroidal antiandrogens | microwave radiation |
| flutamide | |

Combinations of chemotherapeutics are also commonly used standard of care and can be used in combination with a TYR peptide disclosed herein to treat cancers. Such combination therapies include CHOP [cyclophosphamide, doxorubicin hydrochloride (hydroxydaunorubicin), vincristine sulfate (Oncovin), and prednisone], ABVD [doxorubicin (Adriamycin), bleomycin (Blenoxane), vinblastine (Velban) and dacarbazine (DTIC-Dome)], and BEP [bleomycin (Blenoxane), etoposide (Vepesid) and cisplatin (Platinol)].

It is contemplated that the TYR peptide is administered in combination with one or more chemotherapeutic agents or second agents.

In various embodiments, the chemotherapeutic agent is doxorubicin, prednisone, methotrexate, cyclophosphamide, doxorubicin hydrochloride (hydroxydaunorubicin), vincristine sulfate, bleomycin, vinblastine, dacarbazine bleomycin, etoposide, cisplatin, or a combination regimen such as CHOP [cyclophosphamide, doxorubicin hydrochloride (hydroxydaunorubicin), vincristine sulfate (ONCOVIN®), and prednisone], ABVD [doxorubicin (Adriamycin), bleomycin (Blenoxane®), vinblastine (Velban®) and dacarbazine (DTIC-Dome®)], and BEP [bleomycin (Blenoxane®), etoposide (Vepesid®) and cisplatin (Platinol®)].

If TYR peptides are administered in combination with one or more chemotherapeutic agent or second agent, the TYR peptide and or chemotherapeutic or second agent can be administered concurrently or sequentially. Concomitant or concurrent administration of two therapeutic agents does not require that the agents be administered at the same time or by the same route, as long as there is an overlap in the time period during which the agents are exerting their therapeutic effect. Simultaneous or sequential administration is contemplated, as is administration on different days or weeks. It is further contemplated that the therapeutics are administered in a separate formulation and administered concurrently or concomitantly, with concurrently referring to agents given within 30 minutes of each other. Prior administration refers to administration of a therapeutic within the range of one week prior to treatment with a carrier particle, up to 30 minutes before administration of a carrier particle. Subsequent administration is meant to describe administration from 30 minutes after treatment up to one week after administration.

In various embodiments, the one or more chemotherapeutic or second agent is administered prior to, concurrently with or subsequent to administration of the TYR peptide described herein. In various embodiments, the chemotherapeutic or second agent is administered 0.5, 1, 2, 4, 5, 8, 10, 12, 16 or 24 hours prior to administration of the TYR peptide. In various embodiments, the chemotherapeutic or second agent is administered 1, 2, 3, 4, 5, 6, or 7 days prior to administration of the TYR peptide. In various embodiments, the chemotherapeutic or second agent is administered 1, 2, 3, or 4 weeks prior to administration of TYR peptide. In various embodiments, the chemotherapeutic or second agent is administered concurrently with the TYR peptide. In various embodiments, the chemotherapeutic or second agent is administered 0.5 1, 2, 4, 5, 8, 10, 12, 16 or 24 hours subsequent to administration of the TYR peptide. In various embodiments, the chemotherapeutic or second agent is administered 1, 2, 3, 4, 5, 6, or 7 days subsequent to administration of the TYR peptide. In various embodiments, the chemotherapeutic or second agent is administered 1, 2, 3, or 4 weeks subsequent to administration of the TYR peptide.

### Formulation of Pharmaceutical Compositions

To administer peptides of the present disclosure to human or test animals, it is preferable to formulate the peptides in a composition comprising one or more pharmaceutically acceptable carriers. The phrase “pharmaceutically or pharmacologically acceptable” refer to molecular entities and compositions that do not produce allergic, or other adverse reactions when administered using routes well-known in the art, as described below. “Pharmaceutically acceptable carriers” include any and all clinically useful solvents, dispersion media, coatings, antibacterial and antifungal agents, isotonic and absorption delaying agents and the like.

In addition, compounds may form solvates with water or common organic solvents. Such solvates are contemplated as well.

The peptides are administered by any suitable means, including parenteral, subcutaneous, intraperitoneal, intrapulmonary, and intranasal, and, if desired for local treatment, intralesional administration. Parenteral infusions include intravenous, intraarterial, intraperitoneal, intramuscular, intradermal or subcutaneous administration. In various embodiments, the dosing is given by injections, e.g., intravenous or subcutaneous injections, depending in part on whether the administration is brief or chronic. Other administration methods are contemplated, including topical, particularly transdermal, transmucosal, rectal, oral or local administration e.g., through a catheter placed close to the desired site.

Pharmaceutical compositions of the present disclosure containing the peptides described herein as an active ingredient may contain pharmaceutically acceptable carriers or additives depending on the route of administration. Examples of such carriers or additives include water, a pharmaceutical acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Additives used are chosen from, but not limited to, the above or combinations thereof, as appropriate, depending on the dosage form of the present disclosure.

Formulation of the pharmaceutical composition will vary according to the route of administration selected (e.g., solution, emulsion). An appropriate composition comprising a peptide analog described herein to be administered can be prepared in a physiologically acceptable vehicle or carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers.

A variety of aqueous carriers, e.g., sterile phosphate buffered saline solutions, bacteriostatic water, water, buffered water, 0.4% saline, 0.3% glycine, and the like, and may include other proteins for enhanced stability, such as albumin, lipoprotein, globulin, etc., subjected to mild chemical modifications or the like.

Therapeutic formulations of the peptide analogs are prepared for storage by mixing the peptide analog having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulation herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nanoparticles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished by filtration through sterile filtration membranes.

Aqueous suspensions may contain the active compound in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate.

The peptide analogs described herein can be lyophilized for storage and reconstituted in a suitable carrier prior to use. Any suitable lyophilization and reconstitution techniques can be employed. It will be appreciated by those skilled in the art that lyophilization and reconstitution can lead to varying degrees of peptide activity loss and that use levels may have to be adjusted to compensate.

### Kits

As an additional aspect, the disclosure includes kits which comprise one or more compounds or compositions described herein packaged in a manner which facilitates their use to practice methods of the disclosure. In one embodiment, such a kit includes a compound or composition described herein (e.g., a composition comprising a TYR peptide and/or in combination with a second agent), packaged in a container such as a sealed bottle or vessel, with a label affixed to the container or included in the package that describes use of the compound or composition in practicing the method. Preferably, the compound or composition is packaged in a unit dosage form. The kit may further include a device suitable for administering the composition according to a specific route of administration or for practicing a screening assay. Preferably, the kit contains a label that describes use of the inhibitor compositions.

Additional aspects and details of the disclosure will be apparent from the following examples, which are intended to be illustrative rather than limiting.

### Examples

### Example 1—Effects of TYR Peptide on Cell Growth

To determine the effect of TYR peptide on tumor cell growth, rapid cell proliferation assays were carried out on tumor cell lines.

Rapid cell proliferation assay was carried out using a Rapid Cell Proliferation Kit, EMD Biosciences (QIA127) (Galoian K, Tumor Biology 32 (4): 745-51, 2011). Cells were seeded at 5×10⁴ cells/100 μl culture in the multi-well plate and incubated overnight at 37° C. in 5% CO₂ incubator. The actual assay was performed the next day. The rapid cell proliferation assay is based on the activity of mitochondrial enzymes active in viable cells. PRP-1 was added to corresponding wells just after seeding, before overnight incubation. The colorimetric 96-well assay measures colorful product like formazan because of WST-1 tetrazolium salt cleavage by the mitochondrial dehydrogenases. The formazan formation is then quantified by measuring the change in absorbance at 450 nm in a microplate reader. The activity of mitochondrial dehydrogenases is proportional to cell number. No washing, harvesting, or solubilization steps are required. In this series of experiments, PRP-1 was added to corresponding wells immediately after seeding and cell attachment followed by overnight incubation according to the manufacturer's instructions.

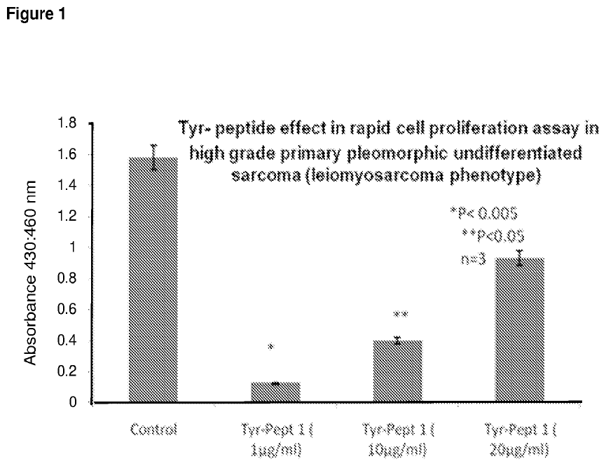

Results show that Tyr peptide reduces proliferation of high grade primary pleomorphic undifferentiated sarcoma (malignant fibrous histiocytoma) of leiomyosarcoma phenotype (FIG. 1). Inhibition with Tyr peptide (1 μg/ml) reached 90% and with Tyr peptide (10 μg/ml)-74% in comparison with not treated control.

### Example 2—Tyr Peptide Efficiently Inhibits Acute Lymphocytic Leukemia (ALL) Cell Line

Acute lymphocytic leukemia (ALL) is a type of cancer of the blood and bone marrow. Acute lymphocytic leukemia is the most common type of cancer in children, and treatments result in a good chance for a cure. Acute lymphocytic leukemia can also occur in adults, though the chance of a cure is greatly reduced. The treatment is mostly chemotherapeutic with toxicity and poor chance of survival in adults (Man L M, et al. Curr Hematol Malig Rep.; 12 (3): 197-206, 2017).

ALL cells were contacted with Tyr peptide and the inhibition of cell growth measured as described above in Example 1.

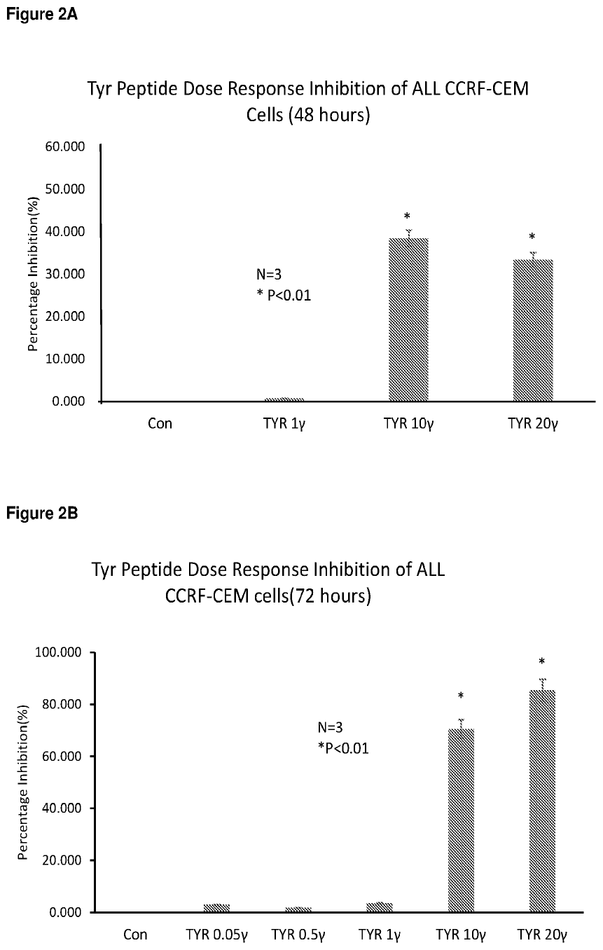

Tyr peptide exhibited 90-80% of growth inhibition in a dose response manner after 72 hours of incubation with 10 and 20 μg/ml of the compound, whereas 40% and 30% of inhibition was registered with the same doses after 48 hours of incubation (FIG. 2).

### Example 3—Anti-Tumor Effects of TYR Peptide In Vivo

Fibrosarcoma cancers respond poorly to conventional chemotherapy and radiotherapy, oftentimes leaving tumor resection as the only option for patients without the promise of a decent survival rate.

This study will evaluate disease progression through clinical signs and animal-relevant responses, tumor size analysis, lung metastasis formation and survival study design. Real-time fluorescence and bioluminescence imaging (IVIS) of both in vivo and ex vivo disease-specific tumor microenvironments and lung metastases will elucidate tremendous information needed to further understand sarcomas. The spheroid approach presents an affordable and innovative alternative that better represents cancer cells in vivo compared to conventional animal 2D cell studies, while promoting an increased growth rate of primary tumors when co-injected with Matrigel (Fridman, et a. Nat Protoc 7:1138-1144 (2012)). Moreover, this approach further enhances tumorigenicity and metastatic spread of xenografted cell lines (Granger et al., supra). This method generates tumors in 2-3 weeks offering a quicker study timeline and avoiding unwarranted animal burden.

Another aspect of this study is based on the fact that chondrosarcomas possess mutations in IDH1/2 enzymes, which catalyze the oxidative decarboxylation of isocitrate, producing a-ketoglutarate (α-KG) and CO₂. Mutant IDH enzymes fail to convert isocitrate into a-ketoglutarate and instead catalyze into oncometabolite (D-2HG), which promotes oncogenesis. The altered epigenetic phenotype has been reported for mutant IDH in leukemias, gliomas, but not in chondrosarcomas. Here experiments may help determine the role of mIDH1 on tumorigenesis of human chondrosarcomas.

The aim of the study is to explore the antitumorigenic effect of neuropeptides PRP-1 and TYR peptide analog (TPA) in vivo, which demonstrated 80-90% inhibition of chondrosarcoma and fibrosarcoma growth under in vitro conditions. A nude mice model was used in which animals receive orthotopic inoculation of highly metastatic JJ012 chondrosarcoma or HT1080 fibrosarcoma tumor cell lines or primary cell lines labeled with extremely red bright fluorescent protein PTDT-N1.

Disease progression is evaluated through clinical signs and animal-relevant responses, tumor size analysis, real-time fluorescence and bioluminescence imaging (IVIS) of both in vivo and ex vivo disease-specific tumor microenvironments and lung metastases.

Subcutaneous implantation of solid tumor implants: The mouse is anesthetized by exposure to 1-5% vaporized isoflurane in 100% oxygen and the depth of anesthesia checked by pedal reflex. The mouse is then transferred to a pre-sterilized heated operating surface and placed dorsal side up for solid tumor implantation or ventral side up for orthotopic mammary fat pad implantation, with the nose fitting inside the nozzle for continued isoflurane delivery. The skin around the incision site is then cleaned with alternating betadine and alcohol three times. Before starting surgery, confirmation that the mouse is fully anesthetized (pedal reflex and respiration) will be performed. For the surgery, a 0.5-1 cm skin incision is made. Using fine surgical forceps and either micro-dissection scissors or toothed forceps, a 5-10 mm subcutaneous pocket is made. Then, a piece of the tumor of approximately 2×2 mm is pushed into the pocket. Staples are used to close the skin incision (and/or Vetbond) to ensure that it does not open. Clips are removed 7-14 days post-injection. For the recovery, the mouse is placed on surgical heat pad until starting to awake, monitoring respiration, while keeping eyes moisture. Buprenorphine (slow releasing form) will be injected subcutaneously (0.5-1 mg/kg) at the time of surgery. Additionally, 0.1 mg/ml Baytril or Gentamicin (0.1-1 mg/ml) is added to the drinking water for mice ad libitum post procedure (replenished every 7 days) to prevent wound infection. After the surgical procedure, the animal is then placed in a clean cage and left undisturbed in a warm, quiet place. The animal is monitored continually until it is conscious and completely recovered from anesthesia.

Solid tumor resection: The mouse is anesthetized by exposure to 1-5% vaporized isoflurane in 100% oxygen and the depth of anesthesia checked by pedal reflex. The mouse is then transferred to a pre-sterilized heated operating surface and placed tumor-bearing side up for solid tumor resection, with the nose fitting inside the nozzle for continued isoflurane delivery. The skin around the incision site is then cleaned with alternating betadine and alcohol three times. Before starting surgery, confirmation that the mouse is fully anesthetized (pedal reflex and respiration) is performed. For the surgery, a 0.5-1 cm skin incision is made on or next to the tumor. Using fine surgical forceps and either micro-dissection scissors or toothed forceps, the tumor is removed trying to remove as much tumor tissue as possible without damaging surrounding normal tissue. Staples are used to close the skin incision (and/or Vetbond) to ensure that it does not open. Clips will be removed 7-14 days post-injection. For the recovery, the mouse will be placed on surgical heat pad until starting to awake, monitoring respiration, while keeping eyes moisture. Buprenorphine (slow releasing form) is injected subcutaneously (0.5-1 mg/kg) at the time of surgery. Additionally, 0.1 mg/ml Baytril or Gentamicin (0.1-1 mg/ml) is added to the drinking water for mice ad libitum post procedure (replenished every 7 days) to prevent wound infection. After the surgical procedure, the animal is then placed in a clean cage and left undisturbed in a warm, quiet place. The animal is monitored continually until it is conscious and completely recovered from anesthesia.

SR buprenorphine and bupivacaine is administered for pain management post-op. Buprenorphine (slow releasing form) is injected subcutaneously (0.5-1 mg/kg) at the time of surgery. Fluid therapy (saline: 0.9% NaCl for ip injection, a maximum of 1 ml/10g) might be administered ip to combat possible dehydration. The animal will be kept warm after surgery by means of a heat pad. Staples or sutures are removed no later than 7-14 days after surgery. For the recovery, the mouse is placed on surgical heat pad until starting to awake, monitoring respiration, while keeping eyes moisture. Additionally, 0.1 mg/ml Baytril or Gentamicin (0.1-1 mg/ml) is added to the drinking water for mice ad libitum post procedure (replenished every 7 days) to prevent infection. After the surgical procedure, the animal is then placed in a clean cage and left undisturbed in a warm, quiet place. The animal is monitored continually until it is conscious and completely recovered from anesthesia. If there are signs of infection, rejection of transplant, or other signs of distress, illness, or infection, the mouse will be euthanized. Euthanasia will be performed consistent with the recommendations of the panel on euthanasia of the American Veterinary Medical Association. Signs include: severe diarrhea, perianal staining, hunched posture, rough haircoat and reluctance to move. If any of these signs are seen post-procedure, the animal will be humanely euthanized.

Monitoring of the solid tumors is as follows (when visible, tumors will be measured by noninvasive imaging or caliper at least weekly). Experimental endpoints depend on the site of transplantation. Mice are euthanized before they reach a tumor volume of 2 cm³ for a single or multiple tumors. The overriding consideration for humane endpoints of these oncological experiments will be the overall health of the animal. Animals are weighed at least once a week for fast growing tumors (reaching endpoint before 3 months after transplantation) and at least once a month for slow growing tumors. Any tumor-bearing animal are euthanized immediately if the tumor impairs ambulation or eating. Tumors located in areas with limited tissue mass are monitored carefully to ensure that the tumor does not interfere with or inhibit movement. Animals bearing tumors in such locations are euthanized before the tumors reach the above-stated dimensions. Animals are euthanized as soon as possible when any of the following clinical signs are shown: (i) Weight loss of 20%; (ii) Mouse displays signs of cachexia and muscle wasting; (iii) Inappetance; (iv) Partial anorexia; (v) Weakness/inability to stand or obtain food/water; (vi) Infection involving any organ system that fails to respond to antibiotic therapy within an appropriate time and is accompanied by systemic signs of illness; (vii) Abnormal appearance; (vii) Abnormal movement or self-mutilation; (ix) Signs of severe organ system dysfunction non-responsive to treatment or with a poor prognosis, as determined by a veterinarian; respiratory distress-dyspnea, agonal breathing and cyanosis.

Tumor-bearing mice are photographed either at the experimental endpoint or using noninvasive imaging technologies to monitor tumor progression. Also contemplated is imaging in vivo for evidence of tumor progression (BLI, IVIS), and observance of clinical signs of metastases (listlessness, weight loss, or hunched posture). Tumors and lungs are harvested to confirm presence of lung metastases and further analyzed: tumors are weighed, photographed, and sectioned into samples designated half for 10% formalin fixation analysis or half for snap-freeze in liquid nitrogen for future analysis.

### Results

Upon initial administration of the peptide to animals, there appears to be no acute toxicity, e.g., change in weight or behavior) with this peptide when treating up to 100 mg/kg.

The IVIS Data showed decreased growth in the TPA Group compared to the Control through Day 14 (IVIS % Growth: (p<0.001); IVIS Raw Data: (p=0.0544)). Ultrasound Measurements were taken at Day 14 to determine if there was a significant difference in Tumor Volume between the TPA and Control groups (p=0.184). The mice were euthanized the following day and tumors were weighed and collected. There was no difference in tumor weights at necropsy.

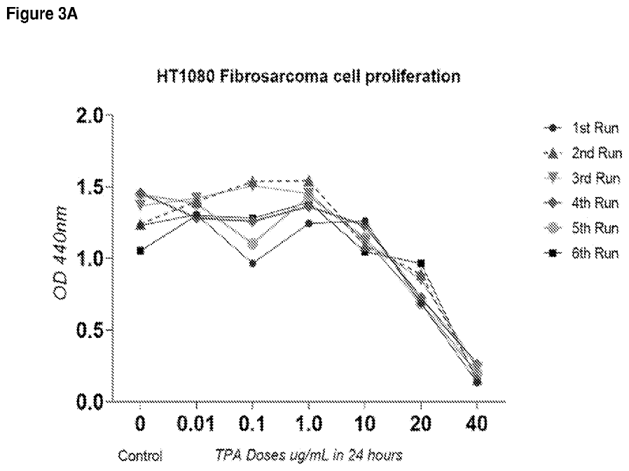

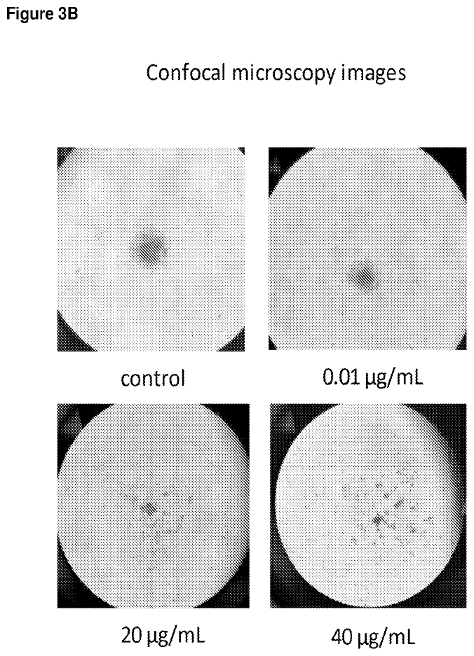

Analysis of anti-proliferative activity in HT1080 cells was examined. FIG. 3A shows a dose response effect with TPA 1 (non-phosphorylated) in human fibrosarcoma, with the maximum effect with 40 μg/ml (IC50=21 μg/mL). FIG. 3B visually demonstrates destruction of cancer stem cell spheroids in dose response manner, with the maximum effect with 40 μg/ml.

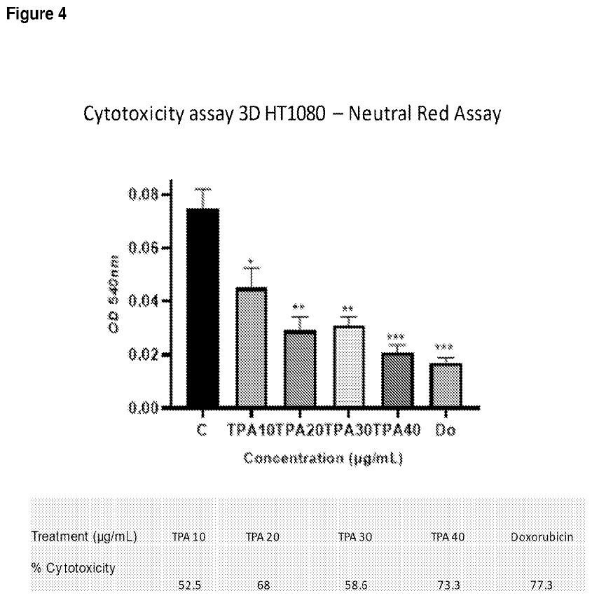

In a neutral red cytotoxicity assay, a dose response decrease in viability of fibrosarcoma spheroids is observed with increased concentration of TPA peptide. The effect of 40 μg/ml is almost that in cytotoxic doxorubicin (DO) (FIG. 4).

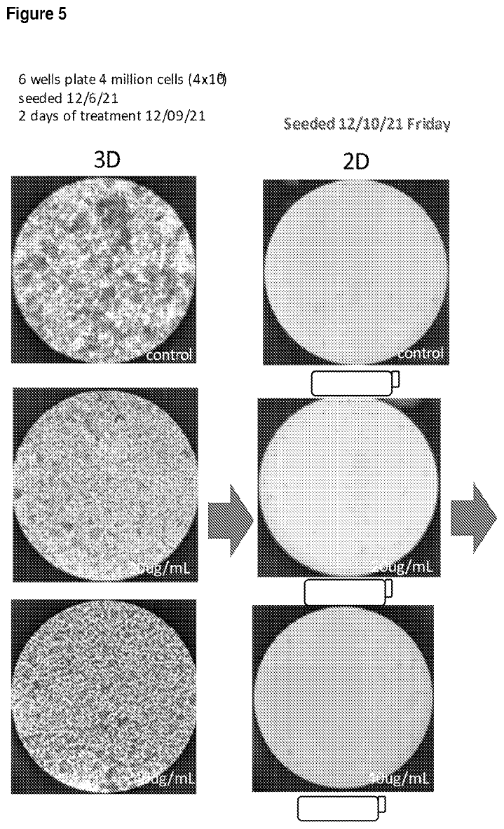

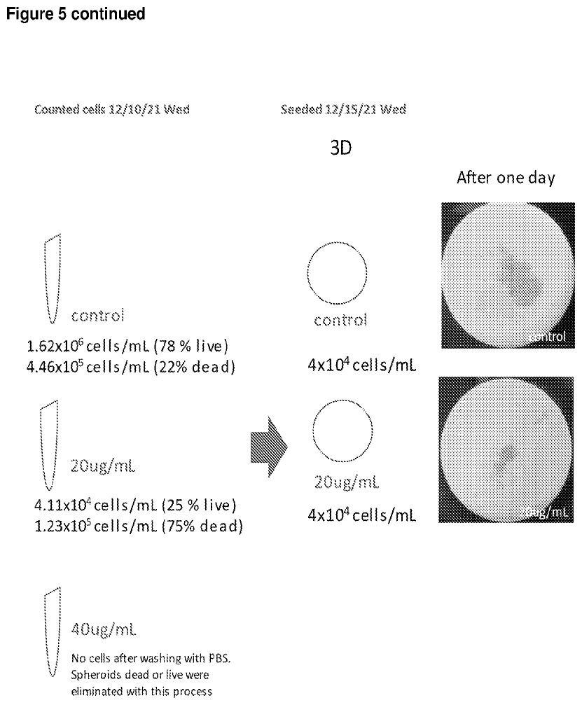

A self-renewal assay showed that spheroids by the administered cells and confirmed their ability to form spheroids again after being dissociated to 2D monolayers. FIG. 5 illustrates the destruction and shrinkage of the spheroid with TPA at 20 μg/ml in comparison to not treated control. Moreover, the shrunken spheroids proved to be non viable by viability assay. Interestingly, treatment with 40 μg/ml of TPA completely prevented spheroid formation.

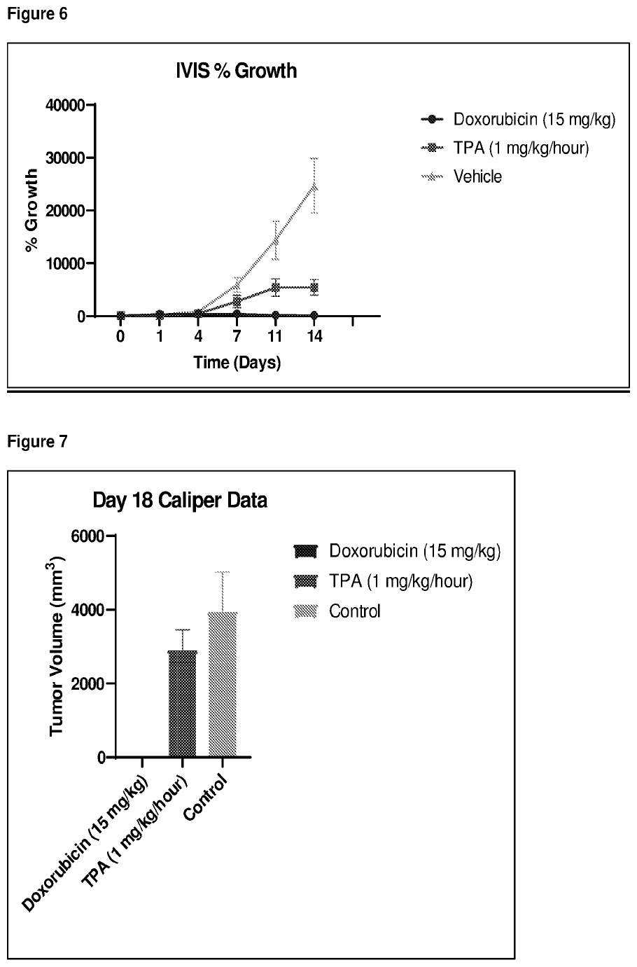

Tumor cell death and necrosis due to hypoxia or drug treatment is indicated early by a reduction in the bioluminescent signal. This cell death might not be accompanied by a reduction in tumor size as measured by physical means. The ability to see early events in tumor necrosis has significant impact on the selection and development of therapeutic agents. Quantitative imaging of tumor growth using IVIS provides precise quantitation and accelerates the experimental process to generate results. IVIS analysis showed decreased growth in the TPA Group compared to the Control through day 14 (FIG. 6). There was also a trend toward decreased tumor volume in TPA treated mice by day 18 (FIG. 7).

These results demonstrate that TYR peptides are effective at reducing tumor cell growth in vitro and in vivo, and are useful to promote cancer stem cell killing.

It is hypothesized that ALDH1A 1 is a target for TPA peptide. There is expanding evidence that ALDH expression increases in response to drug therapy and promotes chemoresistance and survival mechanisms in CSCs. ALDH isozymes have been proposed as new pharmacological targets for the more effective treatment of cancer. Future experiments will undertake to elucidate the effect of TYR peptide on these expression pathways.

### Example 4—Combination Therapy with Doxorubicin

It was next analyzed whether TPA peptides in combination with chemotherapeutics such as doxorubicin would have a combinatorial effect on treatment of tumors in vivo.

HT1080, RFP firefly luciferase (CMV) stable transfected cells (Cellomics Technology (5×10⁵ in 30 μl Matrigel) were implanted intramuscularly in the quadriceps of nu/nu immunocompromised mice. Intravenous injection (tail vein) of Doxorubicin (Doxo) was administered via intravenous tail injection (15 mg/kg.) TPA pumps were installed on the back of the mice subcutaneously, delivering the peptide at a dose rate of 0.025.

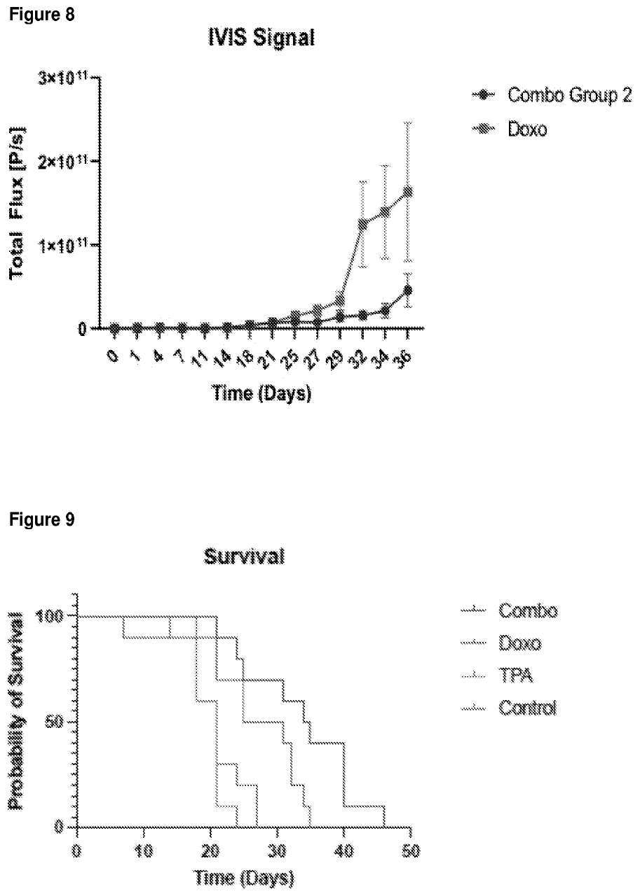

TPA pumps were placed in the animals at day −7 and tumors injected at day −4. TPA was given from day −7 to +7. Relapse was defined as average tumor volume of 400 mm3, day 26. New TPA pumps were transplanted at day +26, and a second doxorubicin treatment begun at day +27. The photon flux from the IVIS is proportional to the number of light emitting cells, which monitors tumor growth. Number of mice used: 6-TPA+Doxo, 10-Doxo only. The results are depicted in (FIG. 8). TPA+Doxo led to a 7-fold decrease and delay in tumor growth, with a manifested delay from day 29-36 in comparison with doxorubicin (Doxo) group. TPA increases overall survival in post chemotherapy relapsed mice, when administered in combination. TPA increases fibrosarcoma mice led to double the survival rate in doxorubicin relapsed mice.

Additional survival experiments were undertaken. HT1080 fibrosarcoma cells were used in this experiment that derived from doxorubicin relapsed mice. The cells from tumor were excised as single cell suspension for quick isolation from tissue for subsequent luminescent labeling, then they were cultured in vitro and re-transplanted. The overall Median Survival advantage (64.3%) in combo treated mice was improved in comparison with Doxo alone (33%) (FIG. 9). Thus, combination treatment of TPA and Doxo led to more than double the survival rate.

It was important to understand whether the survival increase observed in the combination TPA+Doxo treatment is due to a beneficial effect of TPA on Doxo-induced cardiotoxicity. No acute toxicity with this peptide was previously detected up to 100 mg/kg (No change in weight or behavior). Additionally, there appears to be no cumulative toxicity following 14 days of TPA treatment. There was no loss in body weight or effect on grooming mouse posture or behavior and no change in blood count and chemistry indicating no toxicity.

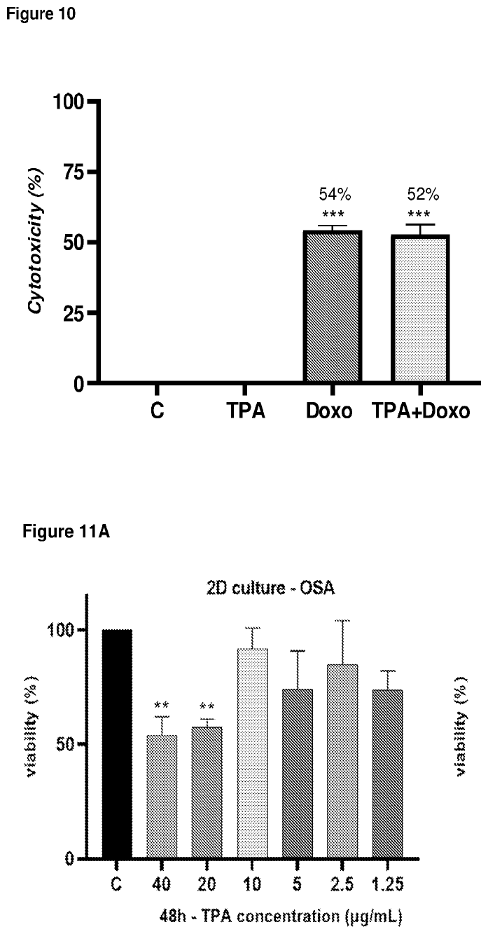

Cardiotoxicity results of TPA, Doxo and TPA+Doxo combo were analyzed in cardiomyocyte cell line AC16. Cells were treated 24 hours with C (control, DMSO 0.5%), TPA (40 μg/mL), Doxo (Doxorubicin, 2 mM), and TPA (40 μg/mL)+Doxo (2 mM). (FIG. 10). After 24 hours of the cardiomyocytes with the corresponding treatments, TPA alone did not show any toxicity within 48 hours and did not rescue Doxorubicin cardio toxicity effect when it was added in combination. Thus, while TPA was not toxic at least 24 hours in culture, TPA did not diminish the cardiotoxicity effect of Doxo. Thus, overall survival increases observed in vivo should be attributed to TPA's ability to target and eliminate cancer stem cells.

### Example 5—Analysis of TPA Peptides in Osteosarcoma Cell Lines

As described above, the antiproliferative effect of TPA was detected in undifferentiated pleomorphic sarcoma (histiocytoma) and acute lymphocytic leukemia (ALL). TPA inhibits osteosarcoma cell growth.

Osteosarcoma is the most common type of bone cancer. Biazzo et al. Acta Orthop Belg. 82 (4): 690-698, 2016. Osteosarcoma is very aggressive bone tumor with a dismal prognosis for poor responders to therapy and for metastasis-presenting patients Odri et al., Cancers. 14:360, 2022. It is rare disease with incidence in USA approximately 1000 cases per year, whereas worldwide statistics indicates at 3.4 cases per 1 million population, Martins-Neves et al., Int J Mol Sci. 23 (19): 11416, 2022:

It has been described that cancer stem cells (CSCs) exist within osteosarcomas (Jubelin, Cancer Drug Resist. 5:184-198, 2022). Local recurrence and metastatic disease occur in 25-50% of surgically-treated patients. The poor response to standard therapy constitutes a major clinical problem: preventing the curing of high-grade osteosarcoma patients. Patients who develop metastatic disease after surgical resection have limited options. Neither the intensification of dose regimens nor the addition of new drugs has significantly modified their clinical outcomes. Zhang et al., J. Bone Oncol. 2018; 12:54-60; Lewis et al., J Natl Cancer Inst.; 99 (2): 112-128 2007.

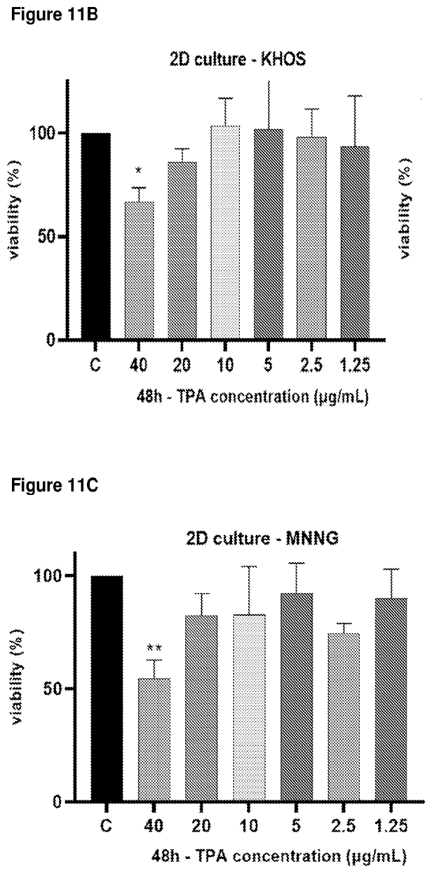

The effects of TPA on three osteosarcoma cell lines OSA, KHOS, MNNG was analyzed (FIG. 11). Rapid cell proliferation was measured by a Rapid Cell Proliferation Kit, EMD Biosciences (QIA127). Cells were seeded at 5×10⁴ cells/100 μl culture in a multi-well plate and incubated overnight at 37° C. in 5% CO₂ incubator. The actual assay was performed the next day. The rapid cell proliferation assay is based on the activity of mitochondrial enzymes active in viable cells. TPA was added to corresponding wells just after seeding, and before overnight incubation. The 96-well assay measures colorimetric product like formazan because of WST-1 tetrazolium salt cleavage by the mitochondrial dehydrogenases. TPA exerted its effect in all cell lines 48 hours after the incubation. In OSA both 20 and 40 μg/ml of TPA inhibited statistically significant growth of this cell line by about 50% (after 48 hrs of incubation), and in MNNG only the highest dose of 40 μg/ml of TPA inhibited growth. Less inhibition (40%) was observed in the KHOS cell line. (FIG. 11)

### Example 6—Analysis of TPA Effect in Different Cancer Types

The effects of TPA on different cancer cell lines was also assessed.

Triple-negative breast cancer (TNBC): Triple-negative breast cancer (TNBC), a specific subtype of basal-like breast cancer (BLBC), does not express estrogen receptor (ER), progesterone receptor (PR), or human epidermal growth factor receptor 2 (HER-2). It has clinical features that include high invasiveness, high metastatic potential, and poor prognosis. Because TNBC tumors lack ER, PR, and HER2 expression, they are not sensitive to endocrine therapy or HER2 treatment, and standardized TNBC treatment regimens are still lacking. TNBC metastasis often involves the brain and visceral organs. Distant metastasis mostly occurs in the 3rd year after diagnosis. The average time to relapse in TNBC patients is only 19-40 months. The mortality rate of TNBC patients within 3 months after recurrence is as high as 75%. Gluz et al., Ann Oncol. 20 (12): 1913-1927, 2009. Therefore, development of new TNBC treatment strategies has become an urgent clinical need.

In a previous publication Galoian KA, (Tumor Biology 2011 32 (4): 745-51) showed that in triple negative aggressive metastatic MDA 231 (ER-) breast cell carcinoma cells maximum inhibition of cell growth with a PRP-1 peptide was observed at 0.5 and 1 μg/ml PRP-1 (71% and 63%, respectively) and inhibition at 10 μg/ml was 44%. There was no inhibitory effect observed on luminal low metastatic T47-D (ER+) cells.

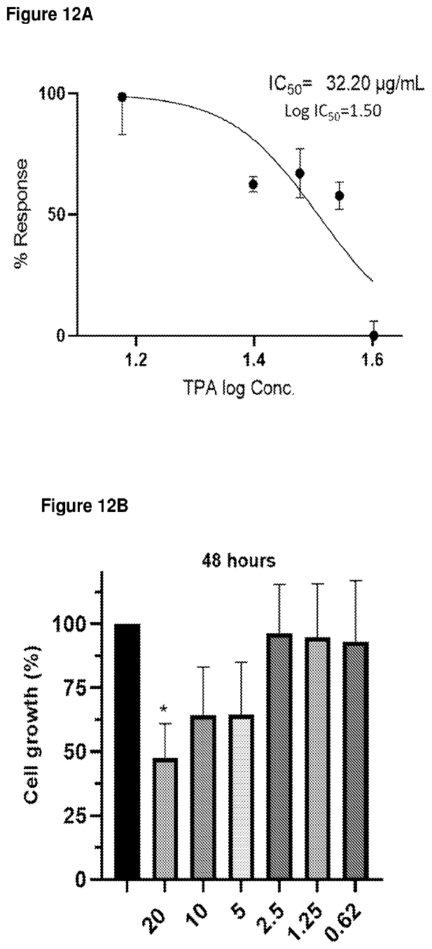

In a new series of experiments, (FIG. 12) the inhibition of breast metastatic carcinoma cells MDA231 (ER-) by TPA peptide was demonstrated using a rapid cell proliferation assay. The cells were exposed for 48h to different concentrations of TPA (1.25-20 μg/mL) and TPA at 20 μg/mL inhibited 47.5% of the cell growth, showing 20 μg/ml of the peptide reduces cell viability. TPA (40 μg/mL) inhibited growth by 36%; (20 μg/mL) inhibited by 38% and (10 μg/mL) inhibited by 32% of the cell growth.

Fibrosarcoma: TPA is a powerful inhibitor of chromatin PRC2 component, (EZH2) and its downstream targets. Polycomb group genes or proteins (PcG) are part of chromatin repressing complex 2 (PRC2), comprised of a set of epigenetic regulators that catalyze specific histone posttranslational modifications. Increasing evidence indicates that enhancer of zeste homolog 2 (EZH2), the catalytic subunit of PRC2, is an epigenetic regulator of chromatin It has been found to increase the proliferation of tumor cells and is highly expressed in CSC population of numerous malignant tumors, contributing also to cancer stem cells maintenance. EZH2 mediates trimethylation of histone H3 lysine 27 (H3K27me3) and exerts inhibition of downstream target genes. Its critical functions, yet to be discovered, most probably extend far beyond its ability to contribute to CSC expansion and maintenance along with its involvement is cell lineage determination. Wen et al., Oncotarget. 8 (23): 37974-37990 2017; Suva, et al., Cancer Res 2009, 69, 9211-9218.

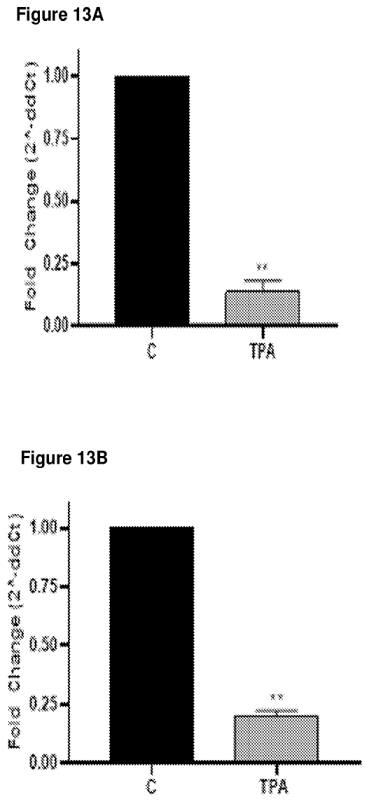

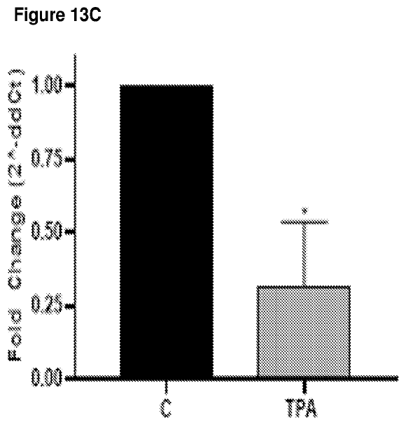

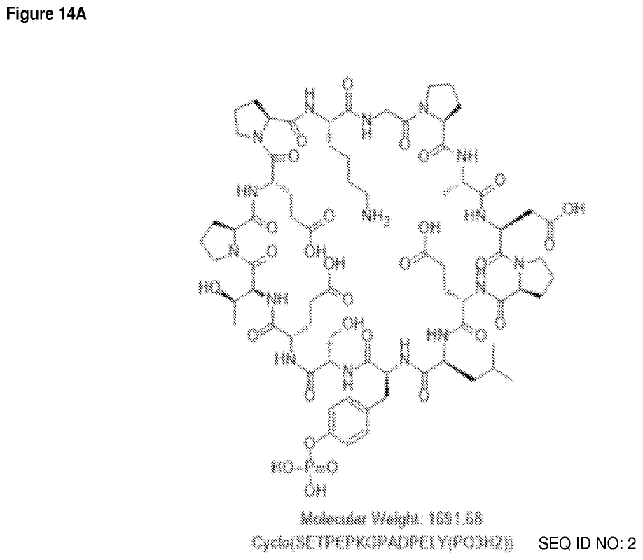

Experiments herein (FIG. 13) indicated there was statistically significant inhibition in 3DHT1080 cell line for EZH2, and its targets, embryonic transcription factor Nanog (Villasante et al., Cell Cycle. 10 (9): 1488-498, 2011), and cancer stem cell marker aldehyde hydrogenase, ALDH1A1 (Li et al., Cancer Prev Res (Phila). 25 (3): 484-491 2012). Aldehyde dehydrogenase (ALDH) is an established marker of CSCs in several neoplasms. Cells expressing high levels of ALDH have been isolated in a number of human sarcoma cell lines, including the human chondrosarcoma SW 1353 cell line (Lohberger et al., PLOS One 7).

FIG. 13 shows that treatment with TPA reduced expression of Nanog by 87%, reduced expression of EZH2 by 80% and reduced expression of ALDHA by 69%. There was also a dose response inhibition of Nanog protein expression in 3D fibrosarcoma cells (HT1080) as TPA (40 μg/ml) inhibited Nanog by 47% in comparison with control in western blot experiments. Gene expression of mRNA sequences coding for Nanog in 3D lysates treated with TPA (20 μg/mL) (7.69 fold decrease) and control; EZH2 in 3D lysates treated with TPA (20 μg/mL), (5 fold decrease), ALDHA in 3D lysates treated with TPA (20 μg/mL), (3.22 fold decrease).

EZH2 can be also upstream (Fillmore et al., Nature. 2015 520 (7546): 239-42) or downstream (Kidder et al., Stem Cells. 2009 February; 27 (2): 317-28) of SWI/SNF chromatin remodeling BAF complex key players Brahma related gene 1 (BRG1) and Brahma (BRM). The original peptide PRP-1 inhibited those units (Moran et al., Oncology reports 44:393-403, 2020 e43664, 2012), and it is hypothesized that TPA will have similar activity.

### Example 7—Cyclization of TYR Peptide

Cyclization is the condensation of NH₂ at the N-terminus and COOH at the C-terminus to form H₂O. It was contemplated that cyclization of TPA may improve its stability.

TYR peptide analog (TPA) was cyclized using standard chemistry. An Fmoc-Pro-CTC Resin with a substitution degree of 0.536 mol/g was soaked for 2 hours in DMF in a reactor. The DMF was drained, and the resin washed. Raw materials were added to the resin according to the equivalent ratio (resin:Fmoc-AA-OH:DIC:HoBt=1:3:3:3), add an appropriate amount of DMF, and nitrogen blown in to react until the ninhydrin is detected to be transparent. After drying, the resin was washed with DMF. The steps were repeated. to obtain Ser (tBu)-Glu (OtBu)-Thr (tBu)-Pro-Glu (OtBu)-Pro-Lys (Boc)-Gly-Pro-CTC Resin (SEQ ID NO: 4).

Raw materials were added in the equivalent ratio (resin:Fmoc-AA-OH:HATU:DIPEA=1:3:2.85:6), and DMF added and resin washed with DMF, DCM, and MeOH for 2 times after draining to obtain Ala-Asp (OtBu)-Pro-Glu (OtBu)-Leu-Tyr (PO3HBzl)-Ser (tBu)-Glu (OtBu)-Thr (tBu)-Pro-Glu (OtBu)-Pro-Lys (Boc)-Gly-Pro-CTC Resin (Compound 1) (SEQ ID NO: 5).

The resin in the reactor was drained, transferred to an Erlenmeyer flask, 1% TFA/DCM solution added, and shaken at temperature for 1 hour.

The resin was filtered by suction filtration, adding an appropriate amount of water to the filtrate, rotary evaporation to remove the solvent, then transferred to a freeze-drying bottle, after freeze-drying, the crude product H-Ala-Asp (OtBu)-Pro-Glu (OtBu)-Leu-Tyr (PO3HBzl)-Ser (tBu)-Glu (OtBu)-Thr (tBu)-Pro-Glu (OtBu)-Pro-Lys (Boc)-Gly-Pro-OH (Compound 2) (SEQ ID NO: 6).

The crude product was dissolved in DCM, adding 3 times the amount of EDC·HCl and 3 times the amount of HOBt, then adding DIPEA to adjust the pH of the solution to 8, and reacted at room temperature for 20 hours. A small amount of water was added, the solvent was removed by rotary evaporation, and then transferred to a freeze-drying bottle, and the crude product Cyclo(Ala-Asp (OtBu)-Pro-Glu (OtBu)-Leu-Tyr (PO3HBzl)-Ser (tBu)-Glu (OtBu)-Thr (tBu)-Pro-Glu (OtBu)-Pro-Lys (Boc)-Gly-Pro) (Compound 3) (SEQ ID NO: 7).

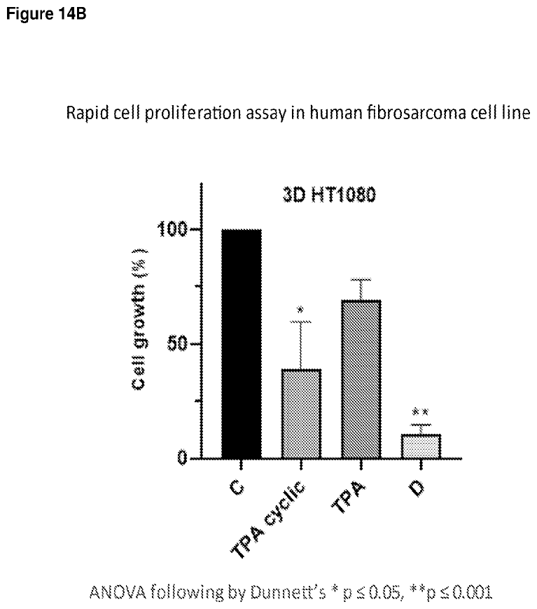

The crude product was transferred to the cutting tube, add solution F (95% TFA+2.5% TIS+2.5% H2O), the temperature controlled on the shaker for 2.5h, adding the cutting solution to 6 times the volume of methyl tert-butyl ether, Centrifugation precipitates and collects solid, and the crude product of precipitation is washed 3 times with methyl tert-butyl ether, and crude product is placed in vacuum desiccator, vacuum-dried overnight, to obtain crude product Cyclo(Ser-Glu-Thr-Pro-Glu-Pro-Lys-Gly-Pro-Ala-Asp-Pro-Glu-Leu-Tyr (PO3H2)) (SEQ ID NO: 8). The crude product was prepared and purified by HPLC, and the final product Cyclo(Ser-Glu-Thr-Pro-Glu-Pro-Lys-Gly-Pro-Ala-Asp-Pro-Glu-Leu-Tyr (PO3H2)) (SEQ ID NO: 8) was isolated (FIG. 14A). The cyclic peptide comprises the same amino acid sequence as the TPA by its structure.

The cyclic version was created to protect the peptide from being degraded by proteases in vivo. The cyclic peptide was tested for its potency in a rapid cell proliferation assay using HT1080 cells as described above using 40 μg/ml TPA or cyclic TPA in the assay. Surprisingly this new formulation is more potent than the non-cyclic TPA peptide as shown in FIG. 14B. inhibiting over 50% of cell growth compared to control, and approximate 1.5 fold improvement over non-cyclic TPA.

Numerous modifications and variations to the disclosure, as set forth in the embodiments and illustrative examples described herein, are expected to occur to those skilled in the art. Consequently only such limitations as appear in the appended claims should be placed on the disclosure.

What is claimed is:

1. A peptide analog of proline rich polypeptide-1 (PRP-1) (AGAPEPAEPAQPGVY, SEQ ID NO: 1) which is a tyrosine peptide (TYR peptide) having an amino acid sequence at least 70% identical to SETPEPKGPADPELY (SEQ ID NO: 2).

2. The TYR peptide of claim 1, wherein the amino acid sequence comprises an amino acid sequence set out in X-X-X-PEP-X-X-PA-X-P-X-X-Y (SEQ ID NO: 3).

3. The TYR peptide of claim 1 or 2, wherein the peptide analog is phosphorylated on the tyrosine residue at the C-terminus.

4. The TYR peptide of any one of claims 1-3, comprising the amino acid sequence SETPEPKGPADPELY (SEQ ID NO: 2) or SETPEPKGPADPELY-P0₃H₂.

5. The TYR peptide of any one of claims 1-4, further comprising a pharmaceutically acceptable excipient, diluent or carrier.

6. The TYR peptide of any one of claims 1-5 that is cyclized.

7. A cyclic TYR peptide having the sequence Cyclo(SETPEPKGPADPELY (PO₃H₂).

8. The cyclic TYR peptide of claim 6 wherein the peptide is cyclized by linking the N-terminal amino acid with the C terminal amino acid.

9. A sterile composition comprising the TYR peptide of any one of claims 1-8.

10. A nucleic acid encoding the TYR peptide of any one of claims 1-8.

11. A method of treating cancer in a subject comprising administering a composition comprising a peptide analog of proline rich polypeptide-1 (PRP-1) which is a tyrosine peptide (TYR peptide) having an amino acid sequence at least 70% identical to SETPEPKGPADPELY (SEQ ID NO: 2).

12. The method of claim 11, wherein the cancer is selected from the group consisting of sarcoma, leukemia and carcinoma.

13. The method of claim 12, wherein the carcinoma is triple negative breast cancer.

14. A method of treating a sarcoma in a subject comprising administering a composition comprising a peptide analog of proline rich polypeptide-1 (PRP-1) which is a tyrosine peptide (TYR peptide) having an amino acid sequence at least 70% identical to SETPEPKGPADPELY (SEQ ID NO: 2).

15. The method of claim 14, wherein the sarcoma is selected from the group consisting of fibrosarcoma, sarcoma of the connective tissue, soft tissue sarcomas, leiomyosarcoma, malignant fibrous histiocytoma (MFH), other undifferentiated pleomorphic sarcomas (PUS) and osteosarcoma.

16. The method of claim 15, wherein the fibrosarcoma is in the subject's thighs, lower leg, knees, arms, trunk, fat, muscles, tendons, nerves, joint tissue, or blood vessels.

17. A method of treating a leukemia in a subject comprising administering a composition comprising a peptide analog of proline rich polypeptide-1 (PRP-1) which is a tyrosine peptide having an amino acid sequence at least 70% identical to SETPEPKGPADPELY (SEQ ID NO: 2).

18. The method of claim 16, wherein the leukemia is selected from the group consisting of acute lymphocytic leukemia (ALL), acute myeloid leukemia (AML), mixed-lineage leukemia (MLL), chronic myeloid (or myelogenous) leukemia (CML), and chronic lymphocytic leukemia (CLL).

19. A method of treating a carcinoma in a subject comprising administering a composition comprising a peptide analog of proline rich polypeptide-1 (PRP-1) which is a tyrosine peptide (TYR peptide) having an amino acid sequence at least 70% identical to SETPEPKGPADPELY (SEQ ID NO: 2).

20. The method of claim 19, wherein the carcinoma is triple negative breast cancer.

21. The method of any one of claims 8-20, wherein the TYR peptide decreases growth and/or proliferation of sarcoma cells, leukemia cells and/or cancer stem cells (CSC).

122. The method of any one of claims 8-21, wherein the TYR peptide is cytotoxic for cancer stem cells (CSC).

23. The method of claim 22, wherein the TYR peptide eliminates 15% or more of cancer stem cells (CSC) in a subject.

24. The method of any one of claims 8-23, wherein the TYR peptide is a cyclic peptide.

25. The method of any one of claims 8-24, wherein the TYR peptide reduces tumor size or tumor volume in the subject.

26. The method of any one of claims 8-25, wherein the TYR peptide reduces incidence of metastasis or reduces incidence of tumor recurrence in the subject.

27. The method of any one of claims 8-26, wherein the subject is non-responsive to previous cancer therapies or developed drug resistance to other cancer therapies.

28. The method of any one of claims 8-29, wherein the TYR peptide is administered with a second agent or standard of care therapy.

29. The method of claim 28, wherein the second agent or standard of care is a chemotherapeutic, a cytotoxic agent, a radionuclide, an immunotherapeutic, radiation therapy, or combinations thereof.

30. The method of claim 29, wherein the chemotherapeutic is selected from the group consisting of doxorubicin, prednisone, methotrexate, cyclophosphamide, doxorubicin hydrochloride (hydroxydaunorubicin), vincristine sulfate, bleomycin, vinblastine, dacarbazine bleomycin, etoposide, cisplatin, CHOP (cyclophosphamide, doxorubicin hydrochloride, vincristine sulfate, and prednisone), ABVD (doxorubicin, bleomycin, vinblastine and dacarbazine), and BEP (bleomycin, etoposide and cisplatin).

31. The method of any one of claims 8-30, wherein the TYR peptide comprises an amino acid sequence set out in X-X-X-PEP-X-X-PA-X-P-X-X-Y (SEQ ID NO: 3).

32. The method of any one of claims 8-31, wherein the TYR peptide is phosphorylated on the C-terminal tyrosine residue.

33. The method of any one of claims 8-32, wherein the TYR peptide comprises the amino acid sequence SETPEPKGPADPELY (SEQ ID NO: 2) or SETPEPKGPADPELY-P0₃H₂.

34. The method of any one of claims 8-32, wherein the TYR peptide comprises the amino acid sequence Cyclo(SETPEPKGPADPELY (PO₃H₂) (SEQ ID NO: 2).
